SEQUENCE LISTING (19 sequences)

SEQ ID NO: 1 - DNA, 1160 bp, Rhodosporidium toruloides
  CDS (1)..(1104): coding for DAAO
atgcactcgcagaagcgcgtcgttgtcctcggatcaggcgttatcggtctgagcagcgccctcatcctcgctcggaagggctacagcgtgcatattctcgcgcgcgacttgccggaggacgtctcgagccagactttcgcttcaccatgggctggcgcgaattggacgcctttcatgacgcttacagacggtcctcgacaagcaaaatgggaagaatcgactttcaagaagtgggtcgagttggtcccgacgggccatgccatgtggctcaaggggacgaggcggttcgcgcagaacgaagacggcttgctcgggcactggtacaaggacatcacgccaaattaccgccccctcccatcttccgaatgtccacctggcgctatcggcgtaacctacgacaccctctccgtccacgcaccaaagtactgccagtaccttgcaagagagctgcagaagctcggcgcgacgtttgagagacggaccgttacgtcgcttgagcaggcgttcgacggtgcggatttggtggtcaacgctacgggacttggcgccaagtcgattgcgggcatcgacgaccaagccgccgagccaatccgcgggcaaaccgtcctcgtcaagtccccatgcaagcgatgcacgatggactcgtccgaccccgcttctcccgcctacatcattccccgaccaggtggcgaagtcatctgcggcgggacgtacggcgtgggagactgggacttgtctgtcaacccagagacggtccagcggatcctcaagcactgcttgcgcctcgacccgaccatctcgagcgacggaacgatcgaaggcatcgaggtcctccgccacaacgtcggcttgcgacctgcacgacgaggcggaccccgcgttgaggcagaacggatcgtcctgcctctcgaccggacaaagtcgcccctctcgctcggcaggggcagcgcacgagcggcgaaggagaaggaggtcacgcttgtgcatgcgtatggcttctcgagtgcgggataccagcagagttggggcgcggcggaggatgtcgcgcagctcgtcgacgaggcgttccagcggtaccacggcgcggcgcgggagtcgaagttgtagggcgggatttgtggctgtattgcgggcatctacaagaaaaaaaaaaaaaaaaa

SEQ ID NO: 2 - protein, 368 aa, Rhodosporidium toruloides
MHSQKRVVVLGSGVIGLSSALILARKGYSVHILARDLPEDVSSQTFASPWAGANWTPFMTLTDGPRQAKWEESTFKKWVELVPTGHAMWLKGTRRFAQNEDGLLGHWYKDITPNYRPLPSSECPPGAIGVTYDTLSVHAPKYCQYLARELQKLGATFERRTVTSLEQAFDGADLVVNATGLGAKSIAGIDDQAAEPIRGQTVLVKSPCKRCTMDSSDPASPAYIIPRPGGEVICGGTYGVGDWDLSVNPETVQRILKHCLRLDPTISSDGTIEGIEVLRHNVGLRPARRGGPRVEAERIVLPLDRTKSPLSLGRGSARAAKEKEVTLVHAYGFSSAGYQQSWGAAEDVAQLVDEAFQRYHGAARESKL

SEQ ID NO: 3 - DNA, 1005 bp, Caenorhabditis elegans
  CDS (1)..(1002): coding for DAAO
atggcaaacataattccgaagattgcaattatcggcgaaggagtcattggatgtacttcagcacttcaaatatcaaaagctataccaaatgcgaaaataactgtgctccacgataaaccatttaaaaaatcgtgcagtgcaggaccagcaggattatttagaatcgattatgaggagaatactgaatacggacgtgcttctttcgcctggttctcacatctctatcgcactacaaaaggatccgaaaccggcgtgaaattagtttctggacatattcaatccgacaacttggagtcattgaagcaacaacaaagagcctatggcgatattgtgtacaactttagattcttggatgatagagaacggctggacatttttcccgaaccatcaaagcactgcattcactacaccgcctacgcatcagaaggtaacaagtacgtgccttatttgaagaatttgctgcttgagcaaaaaatcgagttcaagcaacaagaagtgacgagtttggacgcagtcgccgacgctggttacgatgttattgtaaactgcgcaggcttgtacggtggaaagttggctggtgatgacgatacttgctaccccattagaggagtcattttggaagttgatgcaccatggcacaagcacttcaattatcgagactttactactttcacaattccaaaagagcacagcgtggtggttgggtccaccaagcaggacaatcgatgggatttggagatcaccgacgaggatagaaatgatattttgaaacgatacattgctttacatcctggaatgagagagccaaagattatcaaagaatggtcagcacttcgcccgggacgtaagcatgtcagaattgaagcgcagaagaggacatctgttggaaactcaaaagattatatggttgtgcatcactatggtcacgggagcaacggattcacgttgggttggggaacagcaattgaagcaactaaacttgttaagactgcactaggattataa

SEQ ID NO: 4 - protein, 334 aa, Caenorhabditis elegans
MANIIPKIAIIGEGVIGCTSALQISKAIPNAKITVLHDKPFKKSCSAGPAGLFRIDYEENTEYGRASFAWFSHLYRTTKGSETGVKLVSGHIQSDNLESLKQQQRAYGDIVYNFRFLDDRERLDIFPEPSKHCIHYTAYASEGNKYVPYLKNLLLEQKIEFKQQEVTSLDAVADAGYDVIVNCAGLYGGKLAGDDDTCYPIRGVILEVDAPWHKHFNYRDFTTFTIPKEHSVVVGSTKQDNRWDLEITDEDRNDILKRYIALHPGMREPKIIKEWSALRPGRKHVRIEAQKRTSVGNSKDYMVVHHYGHGSNGFTLGWGTAIEATKLVKTALGL

SEQ ID NO: 5 - DNA, 1186 bp, Nectria haematococca
  CDS (42)..(1124): coding for DAAO
agcgacttgaatttagcgaaaagaacttgtcaaccacaatcatgtccaacacaatcgtcgtcgttggtgccggtgtcattggcttgacgtcggccttgttgctctccaagaacaagggcaacaagatcaccgtcgtggccaagcacatgcccggcgactatgacgttgaatacgcctcgccttttgctggtgccaaccactcccccatggcgacggaagagagcagcgaatgggaacgtcgcacttggtacgagtttaagagactggtcgaggaggtccctgaggccggtgttcatttccagaagtctcgcatccagaggcgcaatgtggacactgaaaaggcgcagaggtctggtttcccagacgccctcttctcgaaagaaccctggttcaagaacatgtttgaggacttccgtgagcagcaccctagcgaggtcatccccggttacgactctggctgcgagttcacatcggtgtgcatcaacacggccatctacctcccctggctcctcggccagtgcatcaagaatggcgtcatcgtcaagcgcgccatcctcaacgacattagcgaggccaagaagctgagccacgcgggcaagacgcccaatatcatcgtcaacgccacgggtctcggctcctacaagctgggcggtgtcgaggacaagaccatggcgcctgcgcggggacagattgtggttgtgcgcaacgagagcagccccatgctcctcacttcaggtgtcgaggacggcggtgctgatgtcatgtacttgatgcagcgagcagctggcggtggcaccatcctgggcggtacctacgacgttggcaactgggagtctcagccagaccccaacatcgcgaatcgcatcatgcagcgcatcgtcgaggtgcggcccgagattgccaacggcaagggcgtcaaggggctgagcgtgatccgacacgccgtcggcatgcggccgtggcgaaaggacggagtcaggatcgaggaggagaagctggatgatgagacttggatcgtgcacaactacggacactctggatggggttaccagggttcgtatggttgtgctgagaatgtagtccagttggttgacaaggtcggcaaggcggccaagtctaagctgtagttgaaaaggcctgaatgagtaatagtaattggatattggaaataccgtatttgccctcg

SEQ ID NO: 6 - protein, 361 aa, Nectria haematococca
MSNTIVVVGAGVIGLTSALLLSKNKGNKITVVAKHMPGDYDVEYASPFAGANHSPMATEESSEWERRTWYEFKRLVEEVPEAGVHFQKSRIQRRNVDTEKAQRSGFPDALFSKEPWFKNMFEDFREQHPSEVIPGYDSGCEFTSVCINTAIYLPWLLGQCIKNGVIVKRAILNDISEAKKLSHAGKTPNIIVNATGLGSYKLGGVEDKTMAPARGQIVVVRNESSPMLLTSGVEDGGADVMYLMQRAAGGGTILGGTYDVGNWESQPDPNIANRIMQRIVEVRPEIANGKGVKGLSVIRHAVGMRPWRKDGVRIEEEKLDDETWIVHNYGHSGWGYQGSYGCAENVVQLVDKVGKAAKSKL

SEQ ID NO: 7 - DNA, 1071 bp, Trigonopsis variabilis
  CDS (1)..(1068): coding for DAAO
atggctaaaatcgttgttattggtgccggtgttgccggtttaactacagctcttcaacttcttcgtaaaggacatgaggttacaattgtgtccgagtttacgcccggtgatcttagtatcggatatacctcgccttgggcaggtgccaactggctcacattttacgatggaggcaagttagccgactacgatgccgtctcttatcctatcttgcgagagctggctcgaagcagccccgaggctggaattcgactcatcagccaacgctcccatgttctcaagcgtgatcttcctaaactggaagttgccatgtcggccatctgtcaacgcaatccctggttcaaaaacacagtcgattctttcgagattatcgaggacaggtccaggattgtccacgatgatgtggcttatctagtcgaatttcgttccgtttgtatccacaccggagtctacttgaactggctgatgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagatgtaccctattcgaggacaagtcgtccttgttcgaaactctcttccttttatggcctccttttccagcactcctgaaaaagaaaatgaagacgaagctctatatatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccaatcctcttacggcatggctgatgaagctgtttcttacgtcgaaagagctcttactcgtccaaacctttag

SEQ ID NO: 8 - protein, 356 aa, Trigonopsis variabilis
MAKIVVIGAGVAGLTTALQLLRKGHEVTIVSEFTPGDLSIGYTSPWAGANWLTFYDGGKLADYDAVSYPILRELARSSPEAGIRLISQRSHVLKRDLPKLEVAMSAICQRNPWFKNTVDSFEIIEDRSRIVHDDVAYLVEFRSVCIHTGVYLNWLMSQCLSLGATVVKRRVNHIKDANLLHSSGSRPDVIVNCSGLFARFLGGVEDKKMYPIRGQVVLVRNSLPFMASFSSTPEKENEDEALYIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 9 - DNA, 1047 bp, Schizosaccharomyces pombe
  CDS (22)..(1041): coding for DAAO
atgactaaggaaaataagccaagagatattgtcatcgttggcgctggcgttattggattgaccactgcttggattctttcagacttgggtcttgctcctcgtattaaggtgattgccaagtatacgcctgaagatcgttctgtagaatacacttccccttgggctggcgcaaatttctgtagcatttctgctactgatgacaatgctttgcgctgggataaaatcacttaccatcgtttcgcctacttggcgaaaactcgtcctgaagcaggaatccgttttgctgatcttcgagaattgtgggagtacgagccgaaacacgacaaaatcagatcctggaatacctatgtcagagatttcaaagttatccctgaaaaagatcttccaggagaatgtatctacggacataaggccaccacctttttaatcaacgctcctcattacttgaattatatgtacaagctgctcattgaagctggcgtcgaatttgaaaagaaagaattgagtcacatcaaagagactgtcgaagaaactccagaagcttcagtagtatttaattgcactggtctctgggcttccaaattgggtggcgttgaagacccggacgtttatccgactcgtggacatgttgttttggttaaggctcctcatgtaacagaaactcgcattttgaatggcaagaactctgatacctatattattcctcgtcccttaaatggtggagtcatttgcggcggtttcatgcaaccaggaaactgggatcgtgaaattcaccctgaagacactttggatatccttaagagaacatcggctttgatgccagaattgttccacggcaagggtccggagggtgctgaaattattcaagaatgtgtcggattccgtccttctcgaaagggtggtgcccgcgtagagcttgatgttgttcccggcacctcagtcccccttgttcatgattacggtgcttctggcacaggataccaagctggttatggtatggctcttgactctgtcatgttggctcttcctaaaatcaaattggcttag

SEQ ID NO: 10 - protein, 340 aa, Schizosaccharomyces pombe
RDIVIVGAGVIGLTTAWILSDLGLAPRIKVIAKYTPEDRSVEYTSPWAGANFCSISATDDNALRWDKITYHRFAYLAKTRPEAGIRFADLRELWEYEPKHDKIRSWNTYVRDFKVIPEKDLPGECIYGHKATTFLINAPHYLNYMYKLLIEAGVEFEKKELSHIKETVEETPEASVVFNCTGLWASKLGGVEDPDVYPTRGHVVLVKAPHVTETRILNGKNSDTYIIPRPLNGGVICGGFMQPGNWDREIHPEDTLDILKRTSALMPELFHGKGPEGAEIIQECVGFRPSRKGGARVELDVVPGTSVPLVHDYGASGTGYQAGYGMALDSVMLALPKIKL

SEQ ID NO: 11 - DNA, 963 bp, Streptomyces coelicolor
  CDS (31)..(957): coding for DAAO
gtggaaaccgaactggatgacgagcgggatggcgaagtcgtcgtggtcggcggcggggtgatcgggctgacgacggccgtcgtcctcgccgagcggggcagacgggtgcggctgtggacccgggagcccgcggagcggaccacctcggtggtagcgggcgggctgtggtggccgtaccggatcgagccggtcgcgctggcccaggcctgggcgctgcgttccctggacgtgtacgaggagctggcggcacggcccgggcagaccggcgtacgcatgctcgaaggggtgctcggcgagaccggcctggacgaggtggacgggtgggccgcggcccggctgccggggctgcgcgcggcgagcgccgccgagtacgccgggacggggctgtgggcgcggctgccgctcatcgacatgtcgacccatctgccgtggctgcgggagcggctgctggccgcgggcggcacggtggaggaccgcgcggtgaccgatctggccgaggcggacgcgccggtggtggtcaactgcaccggcctgggcgcccgggagctggtgccggacccggcggtacggccggtgcgcggacagctggtcgtcgtggagaaccccggcatccacaactggctggtcgcggccgacgcggactccggggagacgacgtacttccttccgcagccgggacggctcctgctgggcggcacggctgaggaggacgcctggtcgaccgagccggacccggaggtcgcggcggccatcgtgcgacggtgcgcggccctgcgtcccgagatcgccggagcgcgggtgctcgcgcacctggtggggctgcggccggcccgggacgcggtccggctggagcgcgggacgctgccggacgggcgccggctggtgcacaactacggtcacggcggcgcgggcgtcaccgtggcctggggctgcgctcaggaggcggcccggctcgcctcctga

SEQ ID NO: 12 - protein, 309 aa, Streptomyces coelicolor
  misc_feature (880)..(936): DAAO signature
GEVVVVGGGVIGLTTAVVLAERGRRVRLWTREPAERTTSVVAGGLWWPYRIEPVALAQAWALRSLDVYEELAARPGQTGVRMLEGVLGETGLDEVDGWAAARLPGLRAASAAEYAGTGLWARLPLIDMSTHLPWLRERLLAAGGTVEDRAVTDLAEADAPVVVNCTGLGARELVPDPAVRPVRGQLVVVENPGIHNWLVAADADSGETTYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLA

SEQ ID NO: 13 - DNA, 1038 bp, Candida boidinii
  CDS (1)..(1035): coding for DAAO
atgggtgatcaaattgttgttcttggttccggtattattggtttatatactacatactgtttaatctatgaggctggatgtgctccagctaaaattactattgttgctgaatttttaccaggtgatcaatctacattatatacatctccatgggcaggtggtaatttttcttgtatttcaccagctgatgatacaacattggcttatgataaattcacatatcttaatttattcaagattcacaaaaaattaggtggaccagaatgtggattagataataagccaagtactgaatattgggatttttatcctggtgatgaaaaagtcaattctttaaaacaatatcttaaagattttaaagttattccaaaatcagaattaccagaaggtgttgaatatggtattagttatactacatggaatttcaactgtcctgttttcttacaaaatatggctaattttttaaataaaagaaatgttaccattattagaaaacatttaacacatatttctcaagcttatttaacagttaatacaaaagttgttttcaactgtacaggtattggtgctgctgatttaggtggtgttaaagatgaaaaagtttatccaactagaggacaagttgttgttgttagagctccacatattcaagaaaataaaatgagatggggtaaagactatgctacttatattattccaagaccatattctaatggtgaattagtcttaggtggtttcttacaaaaggataattggacaggtaatacttttggttttgaaactgatgatattgttagtagaactacatctttattaccaaagattttagatgaaccacttcatattattagagttgcagctggtttaagaccaagtagacatggtggtccaagaattgaagctgaagtttgtgaagaaggtaaattaactattcataattatggtgcttctggatatggttatcaagctggttatggtatgtcttatgaagctgtcaaacttttagttgataaccaaaaagttaaagctaaactttag

SEQ ID NO: 14 - protein, 345 aa, Candida boidinii
MGDQIVVLGSGIIGLYTTYCLIYEAGCAPAKITIVAEFLPGDQSTLYTSPWAGGNFSCISPADDTTLAYDKFTYLNLFKIHKKLGGPECGLDNKPSTEYWDFYPGDEKVNSLKQYLKDFKVIPKSELPEGVEYGISYTTWNFNCPVFLQNMANFLNKRNVTIIRKHLTHISQAYLTVNTKVVFNCTGIGAADLGGVKDEKVYPTRGQVVVVRAPHIQENKMRWGKDYATYIIPRPYSNGELVLGGFLQKDNWTGNTFGFETDDIVSRTTSLLPKILDEPLHIIRVAAGLRPSRHGGPRIEAEVCEEGKLTIHNYGASGYGYQAGYGMSYEAVKLLVDNQKVKAKL

SEQ ID NO: 15 - DNA, 12334 bp, Unknown
  Nucleic acid sequence coding for expression vector STPT GUS Nit-Pdaao
aatattcaaacaaacacatacagcgcgacttatcatggacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcagatctggtaccttgcgccgggtaccccaaactgtctcacgacgttttgaacccagattaccctgttatccctagtcgagcggccgccagtgtgatggatatctgcagaattcgcccttttagatcagcacactggcggccgttactagtggatcaattcactggccgtcgttttacaacgactcagagcttgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatctagagcttgggtcccgcctacaacttcgactcccgcgccgcgccgtggtaccgctggaacgcctcgtcgacgagctgcgcgacatcctccgccgcgccccaactctgctggtatcccgcactcgagaagccatacgcatgcacaagcgtgacctccttctccttcgccgctcgtgcgctgcccctgccgagcgagaggggcgactttgtccggtcgagaggcaggacgatccgttctgcctcaacgcggggtccgcctcgtcgtgcaggtcgcaagccgacgttgtggcggaggacctcgatgccttcgatcgttccgtcgctcgagatggtcgggtcgaggcgcaagcagtgcttgaggatccgctggaccgtctctgggttgacagacaagtcccagtctcccacgccgtacgtcccgccgcagatgacttcgccacctggtcggggaatgatgtaggcgggagaagcggggtcggacgagtccatcgtgcatcgcttgcatggggacttgacgaggacggtttgcccgcggattggctcggcggcttggtcgtcgatgcccgcaatcgacttggcgccaagtcccgtagcgttgaccaccaaatccgcaccgtcgaacgcctgctcaagcgacgtaacggtccgtctctcaaacgtcgcgccgagcttctgcagctctcttgcaaggtactggcagtactttggtgcgtggacggagagggtgtcgtaggttacgccgatagcgccaggtggacattcggaagatgggagggggcggtaatttggcgtgatgtccttgtaccagtgcccgagcaagccgtcttcgttctgcgcgaaccgcctcgtccccttgagccacatggcatggcccgtcgggaccaactcgacccacttcttgaaagtcgattcttcccattttgcttgtcgaggaccgtctgtaagcgtcatgaaaggcgtccaattcgcgccagcccatggtgaagcgaaagtctggctcgagacgtcctccggcaagtcgcgcgcgagaatatgcacgctgtagcccttccgagcgaggatgagggcgctgctcagaccgataacgcctgatccgaggacaacgacgcgcttctgcgagtgcatgggccctcgactagagtcgagatccgatatcgcccgggctcgagtctttgttttttactttggttcatgacactcagagacttgagagaagcaatatatagacttttttttgttttttttttgtggtcacgtttattttcctattggagacggtaacgaagatcgaacctgtggtggaaatgaaacaaggtgggactagcccacgtggtttcttttctctgcattgatttgtttttgttttttttgtaaagttcacatcaaacctactaataattgagaagaaaaataaaatctattgattgattaaaccagccgatgctttatgtctgaatataaaaaagaagtgaaaaccccgtttaagaattacaacggtggtttacaaagtatttggacacaataaatccaaacgaaataaaacaaaatggagaactaccaaataaaaaacaaataaaaaacttaaaagaatttattccattttttttcccgtagaatttattcttttatggattccttaaatccatatttgatgcattttgattcctcataataggtaataatatatactatgttatagatatgtttctaattcgtattaacctacctttttttggtcgtacgattctacctaataatattgaacggaattgatgttttggaccacttagaaagtattttttttttggtttgtcttagctgtatttcattaaatataaatttaaataagaaatgtcataaataaaatttgacgtatagattttttaaatccattttatgttatttaatatttgaaatgtgagtttggctcctatttaatcttaggatgggttaatactaagttttccttaatgaattatctcagagaaactggattaaataaactaaaaaatagatcaatgtgttttggtccggtcaaatatctttggatttactattattggcgaaaagaaagtctcatatagtaaatcatattcctacaagagaaatcaaaatttttgaattaacatggattgtatagtttcttatataaccaattagttcgcatcaagaaaaccaaaccccaattaataatcaaacgggcttggtaggaatatttcattgcagctttcagataaaagaaaaaaacacacactcaagtcttttatttcatctttcttacttgcaggaactcaaattccactttgccacttttctttacaaataaacacaaattgtcaatgaaacgaaatagtctttttatgcaaacactgtttgtcttttttcgatcacgtttctgattgtgacagccatccatatatatagggaatgtaaaacaacaacatgtgaagtcacatatacgtaatggtttagcatagcttctattttcgttgtcaatattagtcattccaaaacatttttaagaaaaataaattaatatatgtatattcttggaactaatgtatgtggaaatacagtaacttaattattaaacattctaaatgcaaatatgcaaagaaaaaaaagaaaagaacacaactgaaatcaaagccagattcataataattggctacatggttgtagaatgtagggtaacacaacatccagaattgaacactcaaattggatgatagatggataatctttagatacaagagaattggttctcttccattattaacgaaaataaagaaaaaaagtttagcataaaagtttgaaactcaacataacattttgaacttgactccttcataggagtgacatgaactgacgaatcacaaccgattacttgtttgagtcatcttccgctttctccaccttcgaaatgaatgtgaccggtttcttcgggtgctcatttacggtcaagtgtaaaacatctggtctcgaggtacctggtagggataacagggtaatctgggttcaaaacgtcgtgagacagtttggtgcaggtcgaaattcgagctcggtacccggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcgccgggtaccgagctcggtagcaattcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgaaatattcccgtgcacttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgtaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgaccaattccacagttttcgcgatccagactgaatgcccacaggccgtcgagttttttgatttcacgggttggggtttctacaggacgtaccatggtcgatcgactctagactagtggatccgatatcgcccgggctcgactctagatgaaatcgaaattcagagttttgatagtgagagcaaagagggacggacttatgaggatttcgagtatttcaagagatggtacttgttgatcggacggctacgatgatctcgatttggttaatccagtatctcgcggtgtatggagttatggtagggttaatggtcaatttcatctaacggtagagaatgatgtaattagataagaatcttgagatactggtttagattggatgagtgtagggtccatcttatcttgataagtggatggtttttagagacacagtgaatattagccaatcgaagttccatatcaccatcatcatctgtataattttgtttttttggaagataataatgattgaaattttggtagattttatttttcattatttaccttgtatgttgagtggtcttcaaattattgaacgtgacagattcacaagaaagtagattttttataaatgaaattttacttattttaaaggtatctctatttaatttcttttgtttatggttgtctgtcagcatttgacttgcagtttcatgctcatagtcatatacgttattctaggcttttttgaatatcttattacttttttcgtaatacaattttataattttatcaaagttatacaactataactaaaattagggttttctacaaaacaaaaaaatcttctaattttttttgttgtagccagtttactcgtaagttacaaaaaaatacaaatgaacccacatgtattatgcgtttaactaggattaccatgtactttcatgtactcaattcaccctatactctttttttttttttttctagttccacccaatctataaaattctgtccatttgaccaaattcaattaatttctgtaattgcgatttaaaattaatattacatgttcactatttctcgatttgagggaacccgagtttaaatatgataaaaatgttgacccatcactacaaatatgttatagtttatacttaatagtggtgtttttggggataattgatgaattaagtaaacatgattcttcttatgaagttgattgagtgattattgtatgtaaacctatgtgattgatgttattggttgattgagtgattattgtattagtatgtaagcaaagatgattgttcttatgaggtaatttgttactcattcatccttttgcatatgagaaattgtgttagcgtacgcaaaacaatagagaacataaaagatatgtgtatttatttaaggtgacttttgttaatgatattgtagtatctatacatttatatataacttgttgaatttgagtataagctatcaggatccgggggatcctctagagtcgaggtaccgagctcgaattcactggccgtcgttttacaacgactcagtactgcttggtaataattgtcattagattgtttttatgcatagatgcactcgaaatcagccaattttagacaagtatcaaacggatgttaattcagtacattaaagacgtccgcaatgtgttattaagttgtctaagcgtcaatttgtttacaccacaatatatcctgccaccagccagccaacagctccccgaccggcagctcggcacaaaatcaccacgcgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagacgccccggtgaatcgtggcaaggggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggcctatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgttccatg

SEQ ID NO: 16 - DNA, 11385 bp, Unknown
  Nucleic acid sequence coding for expression vector STPT GUS ptxA daao
aatattcaaacaaacacatacagcgcgacttatcatggacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcagatctggtaccttgcgccgggtaccccaaactgtctcacgacgttttgaacccagattaccctgttatccctagtcgagcggccgccagtgtgatggatatctgcagaattcgcccttttagatcagcacactggcggccgttactagtggatcaattcactggccgtcgttttacaacgactcagagcttgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatctagagcttgggtcccgcctacaacttcgactcccgcgccgcgccgtggtaccgctggaacgcctcgtcgacgagctgcgcgacatcctccgccgcgccccaactctgctggtatcccgcactcgagaagccatacgcatgcacaagcgtgacctccttctccttcgccgctcgtgcgctgcccctgccgagcgagaggggcgactttgtccggtcgagaggcaggacgatccgttctgcctcaacgcggggtccgcctcgtcgtgcaggtcgcaagccgacgttgtggcggaggacctcgatgccttcgatcgttccgtcgctcgagatggtcgggtcgaggcgcaagcagtgcttgaggatccgctggaccgtctctgggttgacagacaagtcccagtctcccacgccgtacgtcccgccgcagatgacttcgccacctggtcggggaatgatgtaggcgggagaagcggggtcggacgagtccatcgtgcatcgcttgcatggggacttgacgaggacggtttgcccgcggattggctcggcggcttggtcgtcgatgcccgcaatcgacttggcgccaagtcccgtagcgttgaccaccaaatccgcaccgtcgaacgcctgctcaagcgacgtaacggtccgtctctcaaacgtcgcgccgagcttctgcagctctcttgcaaggtactggcagtactttggtgcgtggacggagagggtgtcgtaggttacgccgatagcgccaggtggacattcggaagatgggagggggcggtaatttggcgtgatgtccttgtaccagtgcccgagcaagccgtcttcgttctgcgcgaaccgcctcgtccccttgagccacatggcatggcccgtcgggaccaactcgacccacttcttgaaagtcgattcttcccattttgcttgtcgaggaccgtctgtaagcgtcatgaaaggcgtccaattcgcgccagcccatggtgaagcgaaagtctggctcgagacgtcctccggcaagtcgcgcgcgagaatatgcacgctgtagcccttccgagcgaggatgagggcgctgctcagaccgataacgcctgatccgaggacaacgacgcgcttctgcgagtgcatgggccctcgactagagtcgagatccgatatcgcccgggctcgagtttcgaagattttagtgtaatgtgtgtgctcactactatgaagctttgcacttaaaaaaatagaatgagtgatgaggtttatatggtgaaaaaaactatgaaattttgatattttgatatatctttctcgtgagtcatattcacggaccatgttgcagcaaattggaattaaactattcattttttatgttaaatcattgattgatttttagtgggcctcgttacatattcaagagttagaatgaattcaaacaaactaggccagaaaaaaggatgtggggccatttttttgtgtcttaagaatttgtttatttttttcatggataaggggaatcaatggaaaaagtttgatgtactagaggacatttttttaacatgtagtgacaagtagtgctattattcgacccgtgatgaaaggggcaatcttaatctttttttcataaatctgcacatgtgatgctttaattatgctttagactttgtgctaaactattggtaatttctttttgtaatcgaatcaagtatcttttaaactatgtatgaaatgtgtcatcctaaaaacaacattttgctagttttagactttgatgtttatatgcttaatggaagaagcaatatgttgatgtttattgggtaaaagaaagggacttgattgagtatgtaattgacaactatgattttatattggatttgatattcctaacattaatttaagtgtgtgggtttcaaagcatgttatgctagtgattcttgtgtttgatgcttgaaaaatctacattcatccttgaatggagggacaaactttgaatgacttttgaataggtgtaaaatccaatcctccctcagcttcacaaaaaattgcctcgaggtacctggtagggataacagggtaatctgggttcaaaacgtcgtgagacagtttggtgcaggtcgaaattcgagctcggtacccggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcgccgggtaccgagctcggtagcaattcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgaaatattcccgtgcacttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgtaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgaccaattccacagttttcgcgatccagactgaatgcccacaggccgtcgagttttttgatttcacgggttggggtttctacaggacgtaccatggtcgatcgactctagactagtggatccgatatcgcccgggctcgactctagatgaaatcgaaattcagagttttgatagtgagagcaaagagggacggacttatgaggatttcgagtatttcaagagatggtacttgttgatcggacggctacgatgatctcgatttggttaatccagtatctcgcggtgtatggagttatggtagggttaatggtcaatttcatctaacggtagagaatgatgtaattagataagaatcttgagatactggtttagattggatgagtgtagggtccatcttatcttgataagtggatggtttttagagacacagtgaatattagccaatcgaagttccatatcaccatcatcatctgtataattttgtttttttggaagataataatgattgaaattttggtagattttatttttcattatttaccttgtatgttgagtggtcttcaaattattgaacgtgacagattcacaagaaagtagattttttataaatgaaattttacttattttaaaggtatctctatttaatttcttttgtttatggttgtctgtcagcatttgacttgcagtttcatgctcatagtcatatacgttattctaggcttttttgaatatcttattacttttttcgtaatacaattttataattttatcaaagttatacaactataactaaaattagggttttctacaaaacaaaaaaatcttctaattttttttgttgtagccagtttactcgtaagttacaaaaaaatacaaatgaacccacatgtattatgcgtttaactaggattaccatgtactttcatgtactcaattcaccctatactctttttttttttttttctagttccacccaatctataaaattctgtccatttgaccaaattcaattaatttctgtaattgcgatttaaaattaatattacatgttcactatttctcgatttgagggaacccgagtttaaatatgataaaaatgttgacccatcactacaaatatgttatagtttatacttaatagtggtgtttttggggataattgatgaattaagtaaacatgattcttcttatgaagttgattgagtgattattgtatgtaaacctatgtgattgatgttattggttgattgagtgattattgtattagtatgtaagcaaagatgattgttcttatgaggtaatttgttactcattcatccttttgcatatgagaaattgtgttagcgtacgcaaaacaatagagaacataaaagatatgtgtatttatttaaggtgacttttgttaatgatattgtagtatctatacatttatatataacttgttgaatttgagtataagctatcaggatccgggggatcctctagagtcgaggtaccgagctcgaattcactggccgtcgttttacaacgactcagtactgcttggtaataattgtcattagattgtttttatgcatagatgcactcgaaatcagccaattttagacaagtatcaaacggatgttaattcagtacattaaagacgtccgcaatgtgttattaagttgtctaagcgtcaatttgtttacaccacaatatatcctgccaccagccagccaacagctccccgaccggcagctcggcacaaaatcaccacgcgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagacgccccggtgaatcgtggcaaggggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggcctatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgttccatg

SEQ ID NO: 17 - protein, 19 aa, Unknown
  Consensus motif sequence of D-amino acid oxidase
XXHXYGXXXXGXXXXXGXA

SEQ ID NO: 18 - DNA, 30 bp, Unknown
  PCR primer
attagatcttactactcgaaggacgccatg

SEQ ID NO: 19 - DNA, 30 bp, Unknown
  PCR primer
attagatctacagccacaattcccgcccta

(19) United States
(12) Patent Application Publication
(10) Pub. No.: US 2008/0039328 A1
(43) Pub. Date: Feb. 14, 2008

(54) Post Harvest Control of Genetically Modified Crop Growth Employing D-Amino Acid Compounds

(75) Inventors: Helke Hillebrand, Mannheim (DE); Marcus Ebneth, Berlin (DE); Torgny Nasholm, Holmsund (SE); Oskar Erikson, Holmsund (SE); Magnus Hertzberg, Umea (SE)

Correspondence Address: CONNOLLY BOVE LODGE & HUTZ, LLP, P O BOX 2207, WILMINGTON, DE, 19899, US

(73) Assignees: BASF PLANT SCIENCE GMBH, LUDWIGSHAFEN (DE); SWE TREE TECHNOLOGIES AB, UMEA (SE)

(21) Appl. No.: 10/593,015

(22) PCT Filed: Mar. 15, 2005
(86) PCT No.: PCT/EP05/02735

§ 371 Date: Sep. 15, 2006

(30) Foreign Application Priority Data
Mar. 17, 2004 (EP) 04006377.8

(51) Int. Cl. A01N 33/00; C12N 15/82

(52) U.S. Cl. 504/326; 435/468

(57) ABSTRACT

The invention relates to a method for preventing and/or suppressing growth of transgenic plants comprising a transgenic expression cassette for a D-amino acid oxidase, which are grown on a field, in subsequent seasons among a population of other plants on said field or neighboring fields based on selective killing of the transgenic plants by application of a D-amino acid (e.g. D-isoleucine) which is metabolized by said D-amino acid in said transgenic plants into a phytotoxic compound.

### FIELD OF THE INVENTION

The invention relates to a method for preventing and/or suppressing growth of transgenic plants comprising a transgenic expression cassette for a D-amino acid oxidase, which are grown on a field, in subsequent seasons among a population of other plants on said field or neighboring fields based on selective killing of the transgenic plants by application of a D-amino acid (e.g. D-isoleucine) which is metabolized by said D-amino acid in said transgenic plants into a phytotoxic compound.

### BACKGROUND OF THE INVENTION

An aim of plant biotechnology is the generation of plants with advantageous novel characteristics, for example for increasing agricultural productivity, improving the quality in foodstuffs or for the production of certain chemicals or pharmaceuticals (Dunwell J M (2000) J Exp Bot 51:487-96).

There is however an increased concern about the release of genetically modified crops into the environment. Recent stewardship and labeling laws and regulations require a low percentage of genetically modified material in products to be classified as not comprising genetically modified matter. Even more strict are the requirements for products to be labeled “ecological”.

It is common to plant material that release into the environment is linked with unintended distribution of said material by e.g., cross-pollination. For genetically modified plants this raises the concern that once released it can only hardly be controlled. Once transgenic material was planted on a field, the subsequently grown products will comprise substantial amount of transgenic material.

The methods available so far to control the growth of transgenic crops in subsequent seasons are very limited. There is - for example - the terminator technology which renders the resulting seeds sterile. However, there is strong objection against this technology from farmers since the common farm-saved-seed procedure is impossible based on such crops. Furthermore this technology is limited to sexually propagated crops and cannot be applied to asexually propagated (like e.g, tuber plants like potato). Another alternative is the use of herbicides. There are however no herbicides currently available which selectively kill only the transgenic plant (vice versa herbicides are available with kill only the non-transgenic plant, e.g., glyphosate).

There are some systems known in the art and employed on laboratory scale which allow for selective killing of transgenic organisms (including plants) based on so-called counter-selection marker. These are sequences encoding for enzymes which are able to convert a non-toxic compound into a toxic compound. In consequence, only cells will survive treatment with said non-toxic compound which are lacking said counter-selection marker, thereby allowing for selection of cells which have successfully undergone sequence (e.g., marker) deletion. Typical counter-selection markers known in the art are for example

a) cytosine deaminases (CodA) in combination with 5-fluorocytosine (5-FC) (WO 93/01281; U.S. Pat. No. 5,358,866; Gleave AP et al. (1999) Plant Mol Biol 40(2):223-35; Perera R J et al. (1993) Plant Mol Biol 23(4):793-799; Stougaard J (1993) Plant J 3:755-761); EP-A1 595 837; Mullen C A et al. (1992) Proc Natl Acad Sci USA 89(1):33-37; Kobayashi T et al. (1995) Jpn J Genet 70(3):409-422; Schlaman HRM & Hooykaas PFF (1997) Plant J 11:1377-1385; Xiaohui Wang H et al. (2001) Gene 272(1-2): 249-255; Koprek T et al. (1999) Plant J 19(6):719-726; Gleave AP et al. (1999) Plant Mol Biol 40(2):223-235; Gallego M E (1999) Plant Mol Biol 39(1):83-93; Salomon S & Puchta H (1998) EMBO J 17(20):6086-6095; Thykjaer T et al. (1997) Plant Mol Biol 35(4):523-530; Serino G (1997) Plant J 12(3):697-701; Risseeuw E (1997) Plant J 11(4):717-728; Blanc V et al. (1996) Biochimie 78(6):511-517; Corneille S et al. (2001) Plant J 27:171-178).

b) Cytochrome P-450 enzymes in combination with the sulfonylurea pro-herbicide R7402 (2-methylethyl-2-3-dihydro-N-[(4,6-dimethoxypyrimidine-2-yl)aminocarbonyl]-. 1,2-benzoisothiazol-7-sulfonamid-1,1-dioxide) (O'Keefe D P et al. (1994) Plant Physiol 105:473-482; Tissier A F et al. (1999). Plant Cell 11:1841-1852; Koprek T et al. (1999) Plant J 19(6):719-726; O'Keefe D P (1991) Biochemistry 30(2):447-55).

c) Indoleacetic acid hydrolases like e.g., the tms2 gene product from Agrobacterium tumefaciens in combination with naphthalacetamide (NAM) (Fedoroff N V & Smith D L (1993) Plant J 3:273-289; Upadhyaya N M et al. (2000) Plant Mol Biol Rep 18:227-223; Depicker A G et al. (1988) Plant Cell rep 104:1067-1071; Karlin-Neumannn G A et al. (1991) Plant Cell 3:573-582; Sundaresan V etal. (1995) Gene Develop 9:1797-1810; Cecchini E et al. (1998) Mutat Res 401(1-2):199-206; Zubko E et al. (2000) Nat Biotechnol 18:442-445).

d) Haloalkane dehalogenases (dhlA gene product) from Xanthobacter autotropicus GJ10 in combination with 1,2-dichloroethane (DCE) (Naested H et al. (1999) Plant J 18(5)571-576; Janssen D B et al. (1994) Annu Rev Microbiol 48: 163-191; Janssen D B (1989) J Bacteriol 171(12):6791-9).

e) Thymidine kinases (TK), e.g., from Type 1 Herpes Simplex virus (TK HSV-1), in combination with acyclovir, ganciclovir or 1,2-deoxy-2-fluoro-b-D-arabinofuranosil-5-iodouracile (FIAU) (Czako M & Marton L (1994) Plant Physiol 104:1067-1071; Wigler M et al. (1977) Cell 11(1):223-232; McKnight S L et al. (1980) Nucl Acids Res 8(24):5949-5964; McKnight S L et al. (1980) Nucl Acids Res 8(24):5931-5948; Preston et al. (1981) J Virol 38(2):593-605; Wagner et al. (1981) Proc Natl Acad Sci USA 78(3):1441-1445; St. Clair etal. (1987) Antimicrob Agents Chemother 31(6):844-849).

Several other counter-selection systems are known in the art (see for example international application WO 04/013333; p.13 to 20 for a summary; hereby incorporated by reference). However, these selection systems have at least the following disadvantages:

1. they require use of at least another negative selection marker (e.g., conferring resistance against a herbicide or a antibiotic), which allows for selection of plants which have incorporated the counter-selection marker,

2. the compound used for selection are highly expensive and often only applicable in cell culture or via the medium. None of the above mentioned systems was employed for use as a selective herbicide on the field to control growth of transgenic plants.

WO 03/060133 is describing enzymes like the D-amino acid oxidase from Rhodotorula gracilis. The toxic effect of certain amino acids can—depending on the amino acid—be lowered or increased by metabolization by e.g., a D-amino acid oxidase. There is some teaching about using certain D-amino acids to kill non-transgenic plants and certain D-amino acids to foster growth of transgenic plants, but no teaching for the reverted effects.

As described above there is an unsatisfied demand—especially in the plant biotechnology area—to provide methods and compositions for selectively preventing growth of transgenic plants. This objective has been achieved by the present invention.

### BRIEF DESCRIPTION OF THE INVENTION

Accordingly, a first embodiment of the invention relates to a method for preventing and/or suppressing growth of transgenic plants, which were grown on a field, in subsequent seasons among a population of other plants on said field or neighboring fields comprising the steps of:

i) providing seeds of a transgenic plant comprising at least one first expression cassette comprising a nucleic acid sequence encoding a D-amino acid oxidase operably linked with a promoter allowing expression in plants, in combination with at least one second expression cassette suitable for conferring to said plant an agronomically valuable trait, and

ii) in a first season sowing said seeds on a field, growing said transgenic plants, and harvesting the resulting plant products,

iii) providing at least one compound M, which is non-phytotoxic or moderately phytotoxic against plants not comprising a transgenic expression cassette for a D-amino acid oxidase, wherein said compound M can be metabolized by said D-amino acid oxidase into one or more compound(s) N which are phytotoxic or more phytotoxic than compound M, and

iii) in a subsequent season preventing and/or suppressing growth of said transgenic plants on said field or neighboring fields or areas, where other plants are grown or growing not comprising a transgenic expression cassette for a D-amino acid oxidase, by treating said fields or areas with said compound M in a concentration, which is non-phytotoxic against said other plants, but which is—in consequence of the metabolization into compound(s) N—phytotoxic against said transgenic plants thereby selectively preventing or suppressing growth of said transgenic plants.

In another preferred embodiment the (non-phytotoxic, but metabolizable into phytotoxic) compound M is preferably comprising a D-amino acid structure selected from the group consisting of D-isoleucine, D-valine, D-asparagine, D-leucine, D-lysine, D-proline, and D-glutamine, and derivatives thereof. Preferably, M is comprising and/or consisting of D-isoleucine, D-valine, or derivatives thereof.

There are multiple D-amino acid oxidases known in the art which may be employed within the method of the invention. Preferably, the D-amino acid oxidase expressed from the DNA-construct of the invention has preferably metabolising activity against at least one D-amino acid and comprises a sequences motive having the following consensus sequence:

[LIVM]-[LIVM]-H*-[NHA]-Y-G-x-[GSA]-[GSA]-x-G-x₅-G-
x-A

wherein the amino acid residues given in brackets represent alternative residues for the respective position, x represents any amino acid residue, and indices numbers indicate the respective number of consecutive amino acid residues.

For example the D-amino acid oxidase is described by a sequence of the group consisting of sequences described by GenBank or SwisProt Acc. No. JX0152, O01739, O33145, O35078, O45307, P00371, P14920, P18894, P22942, P24552, P31228, P80324, Q19564, Q28382, Q7PWX4, Q7PWY8, Q7Q7G4, Q7SFW4, Q7Z312, Q82MI8, Q86JV2, Q8N552, Q8P4M9, Q8PG95, Q8R2R2, Q8SZN5, Q8VCW7, Q921M5, Q922Z0, Q95XG9, Q99042, Q99489, Q9C1L2, Q9JXF8, Q9V5P1, Q9VM80, Q9X7P6, Q9Y7N4, Q9Z1M5, Q9Z302, and U60066.

More preferably, the D-amino acid oxidase is selected from the group of amino acid sequences consisting of

a) the sequences described by SEQ ID NO: 2, 4, 6, 8, 10, 12, and 14, and

b) the sequences having a sequence homology of at least 40%, preferably 60%, more preferably 80%, most preferably 95% with a sequence as described by SEQ ID NO: 2, 4, 6, 8, 10, 12, and 14, and

c) the sequences hybridizing under low or high stringency conditions—preferably under high stringency conditions—with a sequence as described by SEQ ID NO: 2, 4, 6, 8, 10, 12, and 14.

Another embodiment of the invention is related to selective herbicidal composition comprising at least one compound M, wherein M is comprising a D-amino acid structure, preferably selected from the group consisting of D-isoleucine, D-valine, D-asparagine, D-leucine, D-lysine, D-proline, and D-glutamine, and derivatives thereof. In a preferred embodiment the selective herbicidal composition comprising at least one compound selected from the group consisting of D-isoleucine, D-valine, and derivatives thereof. An other embodiment of the invention is related to the use of a selective herbicidal composition of the invention to prevent or suppress unwanted growth of transgenic plants.

### GENERAL DEFINITIONS

The teachings, methods, sequences etc. employed and described in the international patent applications WO 03/004659, WO 04/013333, WO 03/060133 are hereby incorporated by reference.

To facilitate understanding of the invention, a number of terms are defined below. It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention which will be limited only by the appended claims. It must be noted that as used herein and in the appended claims, the singular forms “a” and “the” include plural reference unless the context clearly dictates otherwise. Thus, for example, reference to “a vector” is a reference to one or more vectors and includes equivalents thereof known to those skilled in the art, and so forth.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, the word “or” means any one member of a particular list and also includes any combination of members of that list.

“Agronomically valuable trait” include any phenotype in a plant organism that is useful or advantageous for food production or food products, including plant parts and plant products. Non-food agricultural products such as paper, etc. are also included. A partial list of agronomically valuable traits includes pest resistance, vigor, development time (time to harvest), enhanced nutrient content, novel growth patterns, flavors or colors, salt, heat, drought and cold tolerance, and the like. Preferably, agronomically valuable traits do not include selectable marker genes (e. g., genes encoding herbicide or antibiotic resistance used only to facilitate detection or selection of transformed cells), hormone biosynthesis genes leading to the production of a plant hormone (e.g., auxins, gibberilins, cytokinins, abscisic acid and ethylene that are used only for selection), or reporter genes (e.g. luciferase, glucuronidase, chloramphenicol acetyl transferase (CAT, etc.). Such agronomically valuable important traits may include improvement of pest resistance (e.g., Melchers et al. (2000) Curr Opin Plant Biol 3(2):147-52), vigor, development time (time to harvest), enhanced nutrient content, novel growth patterns, flavors or colors, salt, heat, drought, and cold tolerance (e.g., Sakamoto et al. (2000) J Exp Bot 51(342):81-8; Saijo et al. (2000) Plant J 23(3): 319-327; Yeo et al. (2000) Mol Cells 10(3):263-8; Cushman et al. (2000) Curr Opin Plant Biol 3(2):117-24), and the like. Those of skill will recognize that there are numerous polynucleotides from which to choose to confer these and other agronomically valuable traits.

As used herein, the term “amino acid sequence” refers to a list of abbreviations, letters, characters or words representing amino acid residues. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The abbreviations used herein are conventional one letter codes for the amino acids: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine ; S, serine; T, threonine; V, valine; W, tryptophan; Y, tyrosine; Z, glutamine or glutamic acid (see L. Stryer, Biochemistry, 1988, W. H. Freeman and Company, New York. The letter “x” as used herein within an amino acid sequence can stand for any amino acid residue.

The term “nucleotide sequence of interest” refers to any nucleotide sequence, the manipulation of which may be deemed desirable for any reason (e.g., confer improved qualities), by one of ordinary skill in the art. Such nucleotide sequences include, but are not limited to, coding sequences of structural genes (e.g., reporter genes, selection marker genes, oncogenes, drug resistance genes, growth factors, etc.), and noncoding regulatory sequences which do not encode an mRNA or protein product, (e.g., promoter sequence, polyadenylation sequence, termination sequence, enhancer sequence, etc.). A nucleic acid sequence of interest may preferably encode for an agronomically valuable trait.

The term “nucleic acid” refers to deoxyribonucleotides or ribonucleotides and polymers or hybrids thereof in either single-or double-stranded, sense or antisense form. Unless otherwise indicated, a particular nucleic acid sequence also implicitly encompasses conservatively modified variants thereof (e. g., degenerate codon substitutions) and complementary sequences, as well as the sequence explicitly indicated. The term “nucleic acid” is used interchangeably herein with “gene”, “cDNA, “mRNA”, “oligonucleotide,” and “polynucleotide”.

The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid which is transcribed and translated in a sequence-specific manner to produce into a particular polypeptide or protein when placed under the control of appropriate regulatory sequences. The coding region is said to encode such a polypeptide or protein.

A “polynucleotide construct” refers to a nucleic acid at least partly created by recombinant methods. The term “DNA construct” is referring to a polynucleotide construct consisting of deoxyribonucleotides. The construct may be single- or—preferably—double stranded. The construct may be circular or linear.

The skilled worker is familiar with a variety of ways to obtain one of a DNA construct. Constructs can be prepared by means of customary recombination and cloning techniques as are described, for example, in T. Maniatis, E. F. Fritsch and J. Sambrook, Molecular Cloning: A Laboratory Manual, Cold Spring Harbor Laboratory, Cold Spring Harbor, N.Y. (1989), in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley lnterscience (1987).

The term “sense” is understood to mean a nucleic acid having a sequence which is homologous or identical to a target sequence, for example a sequence which binds to a protein transcription factor and which is involved in the expression of a given gene. According to a preferred embodiment, the nucleic acid comprises a gene of interest and elements allowing the expression of the said gene of interest.

The term “antisense” is understood to mean a nucleic acid having a sequence complementary to a target sequence, for example a messenger RNA (mRNA) sequence the blocking of whose expression is sought to be initiated by hybridization with the target sequence.

As used herein, the terms “complementary” or “complementarity” are used in reference to nucleotide sequences related by the base-pairing rules. For example, the sequence 5′-AGT-3′ is complementary to the sequence 5′-ACT-3′. Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. A “complement” of a nucleic acid sequence as used herein refers to a nucleotide sequence whose nucleic acids show total complementarity to the nucleic acids of the nucleic acid sequence.

The term “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

The term “chromosomal DNA” or “chromosomal DNA-sequence” is to be understood as the genomic DNA of the cellular nucleus independent from the cell cycle status. Chromosomal DNA might therefore be organized in chromosomes or chromatids, they might be condensed or uncoiled. An insertion into the chromosomal DNA can be demonstrated and analyzed by various methods known in the art like e.g., polymerase chain reaction (PCR) analysis, Southern blot analysis, fluorescence in situ hybridization (FISH), and in situ PCR.

The term “gene” refers to a coding region operably joined to appropriate regulatory sequences capable of regulating the expression of the polypeptide in some manner. A gene includes untranslated regulatory regions of DNA (e.g., promoters, enhancers, repressors, etc.) preceding (upstream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TM, TAG, TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the nontranslated sequences present on the mRNA transcript). The 5′-flanking region may contain regulatory sequences such as promoters and enhancers which control or influence the transcription of the gene. The 3′-flanking region may contain sequences which direct the termination of transcription, posttranscriptional cleavage and polyadenylation.

The term “expression construct” or “expression construct” as used herein is intended to mean the combination of any nucleic acid sequence to be expressed in operable linkage with a promoter sequence and—optionally—additional elements (like e.g., terminator and/or polyadenylation sequences) which facilitate expression of said nucleic acid sequence.

The term “promoter,” “promoter element,” or “promoter sequence” as used herein, refers to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest (e.g., proximal to the transcriptional start site of a structural gene) whose transcription into mRNA it controls, and provides a site for specific binding by RNA polymerase and other transcription factors for initiation of transcription. A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different coding sequence refers to a coding sequence from a species different from that from which the promoter was derived, or, if from the same species, a coding sequence which is not naturally associated with the promoter (e. g. a genetically engineered coding sequence or an allele from a different ecotype or variety). Suitable promoters can be derived from plants or plant pathogens like e.g., plant viruses.

Promoters may be tissue specific or cell specific. The term “tissue specific” as it applies to a promoter refers to a promoter that is capable of directing selective expression of a nucleotide sequence of interest to a specific type of tissue (e.g., petals) in the relative absence of expression of the same nucleotide sequence of interest in a different type of tissue (e.g., roots). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific”. as applied to a promoter refers to a promoter which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., GUS activity staining (as described for example in Example 7) or immunohistochemical staining. Briefly, tissue sections are embedded in paraffin, and paraffin sections are reacted with a primary antibody which is specific for the polypeptide product encoded by the nucleotide sequence of interest whose expression is controlled by the promoter. A labeled (e.g., peroxidase conjugated) secondary antibody which is specific for the primary antibody is allowed to bind to the sectioned tissue and specific binding detected (e.g., with avidin/biotin) by microscopy. Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. In contrast, a “regulatable” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus.

Where expression of a gene in all tissues of a transgenic plant or other organism is desired, one can use a “constitutive” promoter, which is generally active under most environmental conditions and states of development or cell differentiation (Benfey et al. (1989) EMBO J. 8:2195-2202). The promoter controlling expression of the trait gene and/or selection marker can be constitutive. Suitable constitutive promoters for use in plants include, for example, the cauliflower mosaic virus (CaMV) 35S transcription initiation region (Franck et al. (1980) Cell 21:285-294; Odell et al. (1985) Nature 313:810-812; Shewmaker et al. (1985) Virology 140:281-288; Gardner et al. 1986, Plant Mol. Biol. 6, 221-228), the 19S transcription initiation region (U.S. Pat. No. 5,352,605 and WO 84/02913), and region VI promoters, the 1′-or 2′-promoter derived from T-DNA of Agrobacterium tumefaciens, and other promoters active in plant cells that are known to those of skill in the art. Other suitable promoters include the full-length transcript promoter from Figwort mosaic virus, actin promoters, histone promoters, tubulin promoters, or the mannopine synthase promoter (MAS). Other constitutive plant promoters include various ubiquitin or polyubiquitin promoters derived from, inter alia, Arabidopsis (Sun and Callis (1997) Plant J 11(5): 1017-1027), the mas, Mac or DoubleMac promoters (U.S. Pat. No. 5,106,739; Comai et al. (1990) Plant Mol Biol 15:373-381), the ubiquitin promoter (Holtorf S et al. (1995) Plant Mol Biol 29:637-649) and other transcription initiation regions from various plant genes known to those of skill in the art. Useful promoters for plants also include those obtained from Ti-or Ri-plasmids, from plant cells, plant viruses or other organisms whose promoters are found to be functional in plants. Bacterial promoters that function in plants, and thus are suitable for use in the methods of the invention include the octopine synthetase promoter, the nopaline synthase promoter, and the mannopine synthetase promoter. Suitable endogenous plant promoters include the ribulose-1,6-biphosphate (RUBP) carboxylase small subunit (ssu) promoter, the α-conglycinin promoter, the phaseolin promoter, the ADH promoter, and heatshock promoters. Further preferred constitutive promoters are the nitrilase promoter from Arabidopsis thaliana (WO 03/008596) and the Pisum sativum ptxA promoter (e.g., as incorporated in the construct described by SEQ ID NO: 16; base pair 1866-2728, complementary orientation).

Of course, promoters can regulate expression all of the time in only one or some tissues. Alternatively, a promoter can regulate expression in all tissues but only at a specific developmental time point. As noted above, the excision promoter (i. e., the promoter that is linked to the sequence-specific DNA cleaving polynucleotide) is generally not constitutive, but instead is active for only part of the life cycle or at least one tissue of the transgenic organism. One can use a promoter that directs expression of a gene of interest in a specific tissue or is otherwise under more precise environmental or developmental control. Examples of environmental conditions that may affect transcription by inducible promoters include pathogen attack, anaerobic conditions, ethylene or the presence of light. Promoters under developmental control include promoters that initiate transcription only in certain tissues or organs, such as leaves, roots, fruit, seeds, or flowers, or parts thereof. The operation of a promoter may also vary depending on its location in the genome. Thus, an inducible promoter may become fully or partially constitutive in certain locations.

Examples of tissue-specific plant promoters under developmental control include promoters that initiate transcription only in certain tissues, such as fruit, seeds, flowers, anthers, ovaries, pollen, the meristem, flowers, leaves, stems, roots and seeds. The tissue-specific ES promoter from tomato is particularly useful for directing gene expression so that a desired gene product is located in fruits. See, e. g., Lincoln et al. (1988) Proc Natl Acad Sci USA 84:2793-2797; Deikman et al. (1988) EMBO J 7:3315-3320; Deikman et al. (1992) Plant Physiol 100:2013-2017. Other suitable seed specific promoters include those derived from the following genes: MAC1 from maize (Sheridan et al. (1996) Genetics 142:1009-1020, Cat3 from maize (GenBank No. L05934, Ableretal. (1993) Plant Mol Biol 22:10131-1038, the gene encoding oleosin 18kD from maize (GenBank No. J05212, Lee et al. (1994) Plant Mol Biol 26:1981-1987), viviparous-1 from Arabidopsis (Genbank No. U93215), the gene encoding oleosin from Arabidopsis (Genbank No. Z17657), Atmycl from Arabidopsis (Urao et al. (1996) Plant Mol Biol 32:571-576, the 2s seed storage protein gene family from Arabidopsis (Conceicao et al. (1994) Plant 5:493-505) the gene encoding oleosin 20kD from Brassica napus (GenBank No.-M63985), napin from Brassica napus (GenBank No. J02798, Josefsson et al. (1987) J. Biol. Chem. 262:12196-12201), the napin gene family (e.g., from Brassica napus; Sjodahl et al. (1995) Planta 197:264-271, U.S. Pat. No. 5,608,152; Stalberg K, et al. (1996) L. Planta 199: 515-519), the gene encoding the 2S storage protein from Brassica napus (Dasgupta et al. (1993) Gene 133: 301-302), the genes encoding oleosin A (Genbank No. U09118) and oleosin B (Genbank No. U09119) from soybean, the gene encoding low molecular weight sulphur rich protein from soybean (Choi et al. (1995) Mol Gen Genet 246:266-268), the phaseolin gene (U.S. Pat. No. 5,504,200, Bustos M M et al., Plant Cell. 1989;1(9):839-53), the 2S albumin gene (Joseffson L G et al.(1987) J Biol Chem 262: 12196-12201), the legumin gene (Shirsat A et al. (1989) Mol Gen Genet. 215(2):326-331), the USP (unknown seed protein) gene (Bäumlein H et al. (1991) Mol Gen Genetics 225(3):459-67), the sucrose binding protein gene (WO 00/26388), the legumin B4 gene (LeB4; Bäumlein H et al. (1991) Mol Gen Genet 225:121-128; Baeumlein et al. (1992) Plant J 2(2):233-239; Fiedler U et al. (1995) Biotechnology (N.Y.) 13(10):1090-1093), the Ins Arabidopsis oleosin gene (WO9845461), the Brassica Bce4 gene (WO 91/13980), genes encoding the “high-molecular-weight glutenin” (HMWG), gliadin, branching enzyme, ADP-glucose pyrophosphatase (AGPase) or starch synthase. Furthermore preferred promoters are those which enable seed-specific expression in monocots such as maize, barley, wheat, rye, rice and the like. Promoters which may advantageously be employed are the promoter of the Ipt2 or Ipt1 gene (WO 95/15389, WO 95/23230) or the promoters described in WO 99/16890 (promoters of the hordein gene, the glutelin gene, the oryzin gene, the prolamine gene, the gliadin gene, the zein gene, the kasirin gene or the secalin gene).

Further suitable promoters are, for example, specific promoters for tubers, storage roots or roots such as, for example, the class I patatin promoter (B33), the potato cathepsin D inhibitor promoter, the starch synthase (GBSS1) promoter or the sporamin promoter, and fruit-specific promoters such as, for example, the tomato fruit-specific promoter (EP-A 409 625).

Promoters which are furthermore suitable are those which ensure leaf-specific expression. Promoters which may be mentioned are the potato cytosolic FBPase promoter (WO 98/18940), the Rubisco (ribulose-1,5-bisphosphate carboxylase) SSU (small subunit) promoter or the potato ST-LSI promoter (Stockhaus et al. (1989) EMBO J 8(9):2445-2451). Other preferred promoters are those which govern expression in seeds and plant embryos.

Further suitable promoters are, for example, fruit-maturation-specific promoters such as, for example, the tomato fruit-matiration-specific promoter (WO 94/21794), flower-specific promoters such as, for example, the phytoene synthase promoter (WO 92/16635) or the promoter of the P-rr gene (WO 98/22593) or another node-specific promoter as described in EP-A 249676 may be used advantageously. The promoter may also be a pith-specific promoter, such as the promoter isolated from a plant TrpA gene as described in W0 93/07278. A development-regulated promoter is, inter alia, described by Baerson et al. (Baerson S R, Lamppa G K (1993) Plant Mol Biol 22(2):255-67).

Other preferred promoters are promoters induced by biotic or abiotic stress, such as, for example, the pathogen-inducible promoter of the PRP1 gene (Ward et al., Plant Mol Biol 1993, 22: 361-366), the tomato heat-inducible hsp80 promoter (U.S. Pat. No. 5,187,267), the potato chill-inducible alpha-amylase promoter (WO 96/12814) or the wound-induced pinII promoter (EP375091).

Promoters may also encompass further promoters, promoter elements or minimal promoters capable of modifying the expression-specific characteristics. Thus, for example, the tissue-specific expression may take place in addition as a function of certain stress factors, owing to genetic control sequences. Such elements are, for example, described for water stress, abscisic acid (Lam E and Chua N H (1991) J Biol Chem 266(26):17131-17135) and heat stress (Schoffl F et al. (1989) Molecular & General Genetics 217(2-3):246-53).

The term “operable linkage” or “operably linked” is to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfill its intended function to allow, modify, facilitate or otherwise influence expression of said nucleic acid sequence. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. Operable linkage, and an expression construct, can be generated by means of customary recombination and cloning techniques as described (e.g., in Maniatis 1989; Silhavy 1984; Ausubel 1987; Gelvin 1990). However, further sequences which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of promoter and nucleic acid sequence to be expressed, can exist in a vector-integrated form and be inserted into a plant genome, for example by transformation.

The terms “polypeptide”, “peptide”, “oligopeptide”, “polypeptide”, “gene product”, “expression product” and “protein” are used interchangeably herein to refer to a polymer or oligomer of consecutive amino acid residues.

Preferably, the term “isolated” when used in relation to a nucleic acid , as in “an isolated nucleic acid sequence” refers to a nucleic acid sequence that is identified and separated from at least one contaminant nucleic acid with which it is ordinarily associated in its natural source. Isolated nucleic acid is nucleic acid present in a Form or setting that is different from that in which it is found in nature. In contrast, non-isolated nucleic acids are nucleic acids such as DNA and RNA which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs which encode a multitude of proteins. However, an isolated nucleic acid sequence comprising SEQ ID NO:1 includes, by way of example, such nucleic acid sequences in cells which ordinarily contain SEQ ID NO:1 where the nucleic acid sequence is in a chromosomal or extrachromosomal location different from that of natural cells, or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

As used herein, the term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

The term “wild-type”, “natural” or of “natural origin” means with respect to an organism, polypeptide, or nucleic acid sequence, that said organism is naturally occurring or available in at least one naturally occurring organism which is not changed, mutated, or otherwise manipulated by man.

“Transgene”, “transgenic” or “recombinant” refers to an polynucleotide manipulated by man or a copy or complement of a polynucleotide manipulated by man. For instance, a transgenic expression cassette comprising a promoter operably linked to a second polynucleotide may include a promoter that is heterologous to the second polynucleotide as the result of manipulation by man (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)) of an isolated nucleic acid comprising the expression cassette. In another example, a recombinant expression cassette may comprise polynucleotides combined in such a way that the polynucleotides are extremely unlikely to be found in nature. For instance, restriction sites or plasmid vector sequences manipulated by man may flank or separate the promoter from the second polynucleotide. One of skill will recognize that polynucleotides can be manipulated in many ways and are not limited to the examples above.

The term “transgenic” or “recombinant” when used in reference to a cell refers to a cell which contains a transgene, or whose genome has been altered by the introduction of a transgene. The term “transgenic” when used in reference to a tissue or to a plant refers to a tissue or plant, respectively, which comprises one or more cells that contain a transgene, or whose genome has been altered by the introduction of a transgene. Transgenic cells, tissues and plants may be produced by several methods including the introduction of a “transgene” comprising nucleic acid (usually DNA) into a target cell or integration of the transgene into a chromosome of a target cell by way of human intervention, such as by the methods described herein.

The term “transgene” as used herein refers to any nucleic acid sequence which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence which is naturally found in the cell into which it is introduced so long as it does not contain some modification (e.g., a point mutation, the presence of a selectable marker gene, etc.) relative to the naturally-occurring sequence. The term “heterologous DNA sequence” refers to a nucleotide sequence which is ligated to, or is manipulated to become ligated to, a nucleic acid sequence to which it is not ligated in nature, or to which it is ligated at a different location in nature. Heterologous DNA is not endogenous to the cell into which it is introduced, but has been obtained from another cell. Heterologous DNA also includes an endogenous DNA sequence which contains some modification. Generally, although not necessarily, heterologous DNA encodes RNA and proteins that are not normally produced by the cell into which it is expressed. Examples of heterologous DNA include reporter genes, transcriptional and translational regulatory sequences, selectable marker proteins (e.g., proteins which confer drug resistance), etc. Preferably, the term “transgenic” or “recombinant” with respect to a regulatory sequence (e.g., a promoter of the invention) means that said regulatory sequence is covalently joined and adjacent to a nucleic acid to which it is not adjacent in its natural environment.

The term “foreign gene” refers to any nucleic acid (e.g., gene sequence) which is introduced into the genome of a cell by experimental manipulations and may include gene sequences found in that cell so long as the introduced gene contains some modification (e.g., a point mutation, the presence of a selectable marker gene, etc.) relative to the naturally-occurring gene.

Preferably, the term “transgene” or “transgenic” with respect to, for example, a nucleic acid sequence (or an organism, expression construct or vector comprising said nucleic acid sequence) refers to all those constructs originating by experimental manipulations in which either

a) said nucleic acid sequence, or

b) a genetic control sequence linked operably to said nucleic acid sequence a), for example a promoter, or

c) (a) and (b)

is not located in its natural genetic environment or has been modified by experimental manipulations, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the nucleic acid sequence is preferably retained, at least in part. The environment flanks the nucleic acid sequence at least at one side and has a sequence of at least 50 bp, preferably at least 500 bp, especially preferably at least 1000 bp, very especially preferably at least 5000 bp, in length. A naturally occurring expression construct—for example the naturally occurring combination of a promoter with the corresponding gene —becomes a transgenic expression construct when it is modified by non-natural, synthetic “artificial” methods such as, for example, mutagenization. Such methods have been described (U.S. Pat. No. 5,565,350; WO 00/15815).

“Recombinant” polypeptides or proteins refer to polypeptides or proteins produced by recombinant DNA techniques, i.e., produced from cells transformed by an exogenous recombinant DNA construct encoding the desired polypeptide or protein. Recombinant nucleic acids and polypeptide may also comprise molecules which as such does not exist in nature but are modified, changed, mutated or otherwise manipulated by man.

The term “genetically-modified organism” or “GMO” refers to any organism that comprises transgene DNA. Exemplary organisms include plants, animals and microorganisms.

The terms “heterologous nucleic acid sequence” or “heterologous DNA” are used interchangeably to refer to a nucleotide sequence which is ligated to a nucleic acid sequence to which it is not ligated in nature, or to which it is ligated at a different location in nature. Heterologous DNA is not endogenous to the cell into which it is introduced, but has been obtained from another cell. Generally, although not necessarily, such heterologous DNA encodes RNA and proteins that are not normally produced by the cell into which it is expressed.

The term “cell” or “plant cell” as used herein refers to a single cell. The term “cells” refers to a population of cells. The population may be a pure population comprising one cell type. Likewise, the population may comprise more than one cell type. In the present invention, there is no limit on the number of cell types that a cell population may comprise. The cells may be synchronize or not synchronized. A plant cell within the meaning of this invention may be isolated (e.g., in suspension culture) or comprised in a plant tissue, plant organ or plant at any developmental stage.

The term “organ” with respect to a plant (or “plant organ”) means parts of a plant and may include (but shall not limited to) for example roots, fruits, shoots, stem, leaves, anthers, sepals, petals, pollen, seeds, etc.

The term “tissue” with respect to a plant (or “plant tissue”) means arrangement of multiple plant cells including differentiated and undifferentiated tissues of plants. Plant tissues may constitute part of a plant organ (e.g., the epidermis of a plant leaf) but may also constitute tumor tissues (e.g., callus tissue) and various types of cells in culture (e.g., single cells, protoplasts, embryos, calli, protocorm-like bodies, etc.). Plant tissue may be in planta, in organ culture, tissue culture, or cell culture.

The term “plant” as used herein refers to a plurality of plant cells which are largely differentiated into a structure that is present at any stage of a plant's development. Such structures include one or more plant organs including, but are not limited to, fruit, shoot, stem, leaf, flower petal, etc.

The term “expression” refers to the biosynthesis of a gene product. For example, in the case of a structural gene, expression involves transcription of the structural gene into mRNA and—optionally—the subsequent translation of mRNA into one or more polypeptides.

The term “transformation” as used herein refers to the introduction of genetic material (e.g., a transgene) into a cell. Transformation of a cell may be stable or transient. The term “transient transformation” or “transiently transformed” refers to the introduction of one or more transgenes into a cell in the absence of integration of the transgene into the host cell's genome. Transient transformation may be detected by, for example, enzyme-linked immunosorbent assay (ELISA) which detects the presence of a polypeptide encoded by one or more of the transgenes. Alternatively, transient transformation may be detected by detecting the activity of the protein (e.g., β-glucuronidase) encoded by the transgene (e.g., the uid A gene) as demonstrated herein [e.g., histochemical assay of GUS enzyme activity by staining with X-gluc which gives a blue precipitate in the presence of the GUS enzyme; and a chemiluminescent assay of GUS enzyme activity using the GUS-Light kit (Tropix)]. The term “transient transformant” refers to a cell which has transiently incorporated one or more transgenes. In contrast, the term “stable transformation” or “stably transformed” refers to the introduction and integration of one or more transgenes into the genome of a cell, preferably resulting in chromosomal integration and stable heritability through meiosis. Stable transformation of a cell may be detected by Southern blot hybridization of genomic DNA of the cell with nucleic acid sequences which are capable of binding to one or more of the transgenes. Alternatively, stable transformation of a cell may also be detected by the polymerase chain reaction of genomic DNA of the cell to amplify transgene sequences. The term “stable transformant” refers to a cell which has stably integrated one or more transgenes into the genomic DNA. Thus, a stable transformant is distinguished from a transient transformant in that, whereas genomic DNA from the stable transformant contains one or more transgenes, genomic DNA from the transient transformant does not contain a transgene. Transformation also includes introduction of genetic material into plant cells in the form of plant viral vectors involving epichromosomal replication and gene expression which may exhibit variable properties with respect to meiotic stability.

The terms “infecting” and “infection” with a bacterium refer to co-incubation of a target biological sample, (e.g., cell, tissue, etc.) with the bacterium under conditions such that nucleic acid sequences contained within the bacterium are introduced into one or more cells of the target biological sample.

The term “Agrobacterium” refers to a soil-borne, Gram-negative, rod-shaped phytopathogenic bacterium which causes crown gall. The term “Agrobacterium” includes, but is not limited to, the strains Agrobacterium tumefaciens, (which typically causes crown gall in infected plants), and Agrobacterium rhizogenes (which causes hairy root disease in infected host plants). Infection of a plant cell with Agrobacterium generally results in the production of opines (e.g., nopaline, agropine, octopine etc.) by the infected cell. Thus, Agrobacterium strains which cause production of nopaline (e.g., strain LBA4301, C58, A208) are referred to as “nopaline-type” Agrobacteria; Agrobacterium strains which cause production of octopine (e.g., strain LBA4404, Ach5, B6) are referred to as “octopine-type” Agrobacteria; and Agrobacterium strains which cause production of agropine (e.g., strain EHA105, EHA101, A281) are referred to as “agropine-type” Agrobacteria.

The terms “bombarding, “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, the contents of which are herein incorporated by reference), and are commercially available (e.g., the helium gas-driven microprojectile accelerator (PDS-1000/He) (BioRad).

The term “microwounding” when made in reference to plant tissue refers to the introduction of microscopic wounds in that tissue. Microwounding may be achieved by, for example, particle bombardment as described herein.

The terms “homology” or “identity” when used in relation to nucleic acids refers to a degree of complementarity. Homology or identity between two nucleic acids is understood as meaning the identity of the nucleic acid sequence over in each case the entire length of the sequence, which is calculated by comparison with the aid of the program algorithm GAP (Wisconsin Package Version 10.0, University of Wisconsin, Genetics Computer Group (GCG), Madison, USA) with the parameters being set as follows:

| | |
|---|---|
| Gap Weight: 12 | Length Weight: 4 |
| Average Match: 2,912 | Average Mismatch: −2,003 |

For example, a sequence with at least 95% homology (or identity) to the sequence SEQ ID NO. 1 at the nucleic acid level is understood as meaning the sequence which, upon comparison with the sequence SEQ ID NO. 1 by the above program algorithm with the above parameter set, has at least 95% homology. There may be partial homology (i.e., partial identity of less then 100%) or complete homology (i.e., complete identity of 100%).

Alternatively, a partially complementary sequence is understood to be one that at least partially inhibits a completely complementary sequence from hybridizing to a target nucleic acid and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe (i.e., an oligonucleotide which is capable of hybridizing to another oligonucleotide of interest) will compete for and inhibit the binding (i.e., the hybridization) of a completely homologous sequence to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe which can hybridize to either or both strands of the double-stranded nucleic acid sequence under conditions of low stringency as described infra. When used in reference to a single-stranded nucleic acid sequence, the term “substantially homologous” refers to any probe which can hybridize to the single-stranded nucleic acid sequence under conditions of low stringency as described infra.

The term “hybridization” as used herein includes “any process by which a strand of nucleic acid joins with a complementary strand through base pairing.” (Coombs 1994). Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids.

As used herein, the term “Tm” is used in reference to the “melting temperature.” The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41 (% G+C), when a nucleic acid is in aqueous solution at 1 M NaCl [see e.g., Anderson and Young, Quantitative Filter Hybridization, in Nucleic Acid Hybridization (1985)]. Other references include more sophisticated computations which take structural as well as sequence characteristics into account for the calculation of Tm.

Low stringency conditions when used in reference to nucleic acid hybridization comprise conditions equivalent to binding or hybridization at 68° C. in a solution consisting of 5×SSPE (43.8 g/L NaCl, 6.9 g/L NaH₂PO₄.H₂O and 1.85 g/L EDTA, pH adjusted to 7.4 with NaOH), 1% SDS, 5× Denhardt's reagent [50× Denhardt's contains the following per 500 mL: 5 g Ficoll (Type 400, Pharmacia), 5 g BSA (Fraction V; Sigma)] and 100 μg/mL denatured salmon sperm DNA followed by washing in a solution comprising 0.2×SSPE, and 0.1% SDS at room temperature when a DNA probe of about 100 to about 1000 nucleotides in length is employed.

High stringency conditions when used in reference to nucleic acid hybridization comprise conditions equivalent to binding or hybridization at 68° C. in a solution consisting of 5×SSPE, 1% SDS, 5× Denhardt's reagent and 100 μg/mL denatured salmon sperm DNA followed by washing in a solution comprising 0.1×SSPE, and 0.1% SDS at 68° C. when a probe of about 100 to about 1000 nucleotides in length is employed.

The term “equivalent” when made in reference to a hybridization condition as it relates to a hybridization condition of interest means that the hybridization condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 80% to 90% homology to the first nucleic acid sequence, then another hybridization condition is said to be equivalent to the hybridization condition of interest if this other hybridization condition also results in hybridization of the first nucleic acid sequence with the other nucleic acid sequences that have from 80% to 90% homology to the first nucleic acid sequence.

When used in reference to nucleic acid hybridization the art knows well that numerous equivalent conditions may be employed to comprise either low or high stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of either low or high stringency hybridization different from, but equivalent to, the above-listed conditions. Those skilled in the art know that whereas higher stringencies may be preferred to reduce or eliminate non-specific binding, lower stringencies may be preferred to detect a larger number of nucleic acid sequences having different homologies.

### DETAILED DESCRIPTION OF THE INVENTION

Accordingly, a first embodiment of the invention relates to a method for preventing and/or suppressing growth of transgenic plants, which were grown on a field, in subsequent seasons among a population of other plants on said field or neighboring fields comprising the steps of:

i) providing seeds of a transgenic plant comprising at least one first expression cassette comprising a nucleic acid sequence encoding a D-amino acid oxidase operably linked with a promoter allowing expression in plants, in combination with at least one second expression cassette suitable for conferring to said plant an agronomically valuable trait, and

ii) in a first season sowing said seeds on a field, growing said transgenic plants, and harvesting the resulting plant products,

iii) providing at least one compound M, which is non-phytotoxic or moderately phytotoxic against plants not comprising a transgenic expression cassette for a D-amino acid oxidase, wherein said compound M can be metabolized by said D-amino acid oxidase into one or more compound(s) N which are phytotoxic or more phytotoxic than compound M, and

iii) in a subsequent season preventing and/or suppressing growth of said transgenic plants on said field or neighboring fields or areas, where other plants are grown or growing not comprising a transgenic expression cassette for a D-amino acid oxidase, by treating said fields or areas with said compound M in a concentration, which is non-phytotoxic against said other plants, but which is—in consequence of the metabolization into compound(s) N—phytotoxic against said transgenic plants thereby selectively preventing or suppressing growth of said transgenic plants.

This invention discloses the use of D-amino acid oxidases (DAAO, EC 1.4.3.3) for controlling growth of transgenic plants. DAAO marker can be employed for both negative selection and counter-selection, depending on the substrate used. DAAO catalyzes the oxidative deamination of a range of D-amino acids (Alonso J et al. (1998) Microbiol. 144, 1095-1101). Thus, the D-amino acid oxidase constitutes a dual-function marker. The marker has been successfully established in Arabidopsis thaliana, and proven to be versatile, rapidly yielding unambiguous results, and allowing selection immediately after germination (WO 03/060133)

Many prokaryotes and eukaryotes metabolize D-amino acids (Pilone M S (2000) Cell. Mol. Life. Sci. 57, 1732-174), but current information suggests that D-amino acid metabolism is severely restricted in plants. However, studies of amino acid transporters in plants have shown that several of these proteins may mediate the transport of both L- and D-enantiomers of amino acids, although the latter usually at lower rates (Frommer W B et al. (1995) Proc. Natl. Acad. Sci. USA 92, 12036-12040; Boorer K J et al. (1996) J. Biol. Chem. 271, 2213-22203). These findings imply that plants absorb D-amino acids but metabolize few if any D-amino acids. D-amino acid catabolism follows several routes, one of the most common being oxidative deamination (Pilone M S (2000) Cell. Mol. Life. Sci. 57, 1732-1742). The natural occurrence of D-amino acids in plants is generally low, with measurable levels of D-alanine, D-serine, D-glutamine and D-asparagine but no detectable levels of D-valine and D-isoleucine (Bruckner H & Westhauser T (2003) Amino acids 24, 43-55). Hence, the amount and nature of substrates that DAAO may engage under natural conditions would not cause negative effects on plants.

In another preferred embodiment the second (non-phytotoxic, but metabolizable into phytotoxic) compound M is preferably selected from the group consisting of D-isoleucine, D-valine, D-asparagine, D-leucine, D-lysine, D-proline, and D-glutamine, and derivatives thereof. In contrast to D-amino acids like D-serine and D-alanine, other D-amino acids like D-valine and D-isoleucine, which are not toxic to wild-type plants, have a strong negative influence on the growth of plants expressing DAAO (FIG. 4c,d). The findings that DAAO expression mitigated the toxicity of D-serine and D-alanine, but induced metabolic changes that made D-isoleucine and D-valine toxic, demonstrate that the enzyme could provide a substrate-dependent, dual-function, selectable marker in plants. Selection is based on differences in the toxicity of different D-amino acids and their metabolites to plants. Thus, D-alanine and D-serine are toxic to plants, but are metabolized by DAAO into nontoxic products, whereas D-isoleucine and D-valine have low toxicity, but are metabolized by DAAO into the toxic keto acids 3-methyl-2-oxopentanoate and 3-methyl-2-oxobutanoate, respectively. Hence, both positive and negative selection is possible with the same marker gene, which is therefore considered a dual-function marker.

It is an additional advantage of the invention that the D-amino acid oxidase can not only be employed to prevent or suppress growth of transgenic plants but—due to its functionality as a dual-function marker—can also be utilized during the transformation procedure as a negative selection marker for the production of the transgenic plant. This makes incorporation of additional marker sequences (e.g., for antibiotic or herbicide resistance) oblivious. For its use as a negative selection marker for example D-alanine, D-serine, and derivatives thereof may be employed. The toxicity of D-amino acids like e.g., D-serine and D-alanine can be alleviated by the insertion of a gene encoding an enzyme that metabolizes D-amino acids (e.g., the dao1 gene from the yeast Rhodotorula gracilis). Exposure of this transgenic plant to D-alanine or D-serine showed that it could detoxify both of these D-amino acids.

### I. The D-amino Acid Oxidase Marker of the Invention

The term D-amino acid oxidase (abbreviated DAAO, DAMOX, or DAO) is referring to the enzyme coverting a D-amino acid into a 2-oxo acid, by—preferably—employing Oxygen (O₂) as a substrate and producing hydrogen peroxide (H₂O₂) as a co-product (Dixon M & Kleppe K. (1965) Biochim. Biophys. Acta 96:357-367; Dixon M & Kleppe K Biochim. Biophys. Acta 96 (1965) 368-382; Dixon M & Kleppe Biochim. Biophys. Acta 96 (1965) 383-389; Massey V et al. (1961) Biochim. Biophys. Acta 48:1-9. Meister A & Wellner D Flavoprotein amino acid oxidase. In: Boyer, P. D., Lardy, H. and Myrbäck, K. (Eds.), The Enzymes, 2nd ed., vol. 7, Academic Press, N.Y., 1963, p. 609-648.)

DAAO can be described by the Nomenclature Committee of the International Union of Biochemistry and Molecular Biology (IUBMB) with the EC (Enzyme Commission) number EC 1.4.3.3. Generally an DAAO enzyme of the EC 1.4.3.3. class is an FAD flavoenzyme that catalyzes the oxidation of neutral and basic D-amino acids into their corresponding keto acids. DAAOs have been characterized and sequenced in fungi and vertebrates where they are known to be located in the peroxisomes. The term D-amino oxidase further comprises D-aspartate oxidases (EC 1.4.3.1) (DASOX) )(Negri A et al. (1992) J Biol Chem. 267:11865-11871), which are enzymes structurally related to DAAO catalyzing the same reaction but active only toward dicarboxylic D-amino acids. Within this invention DAAO of the EC 1.4.3.3. class are preferred.

In DAAO, a conserved histidine has been shown (Miyano M et al. (1991) J Biochem 109:171-177) to be important for the enzyme's catalytic activity. In a preferred embodiment of the invention a DAAO is referring to a protein comprising the following consensus motive:

[LIVM]-[LIVM]-H*-[NHA]-Y-G-x-[GSA]-[GSA]-x-G-x₅-G-
x-A

wherein the amino acid residues given in brackets represent alternative residues for the respective position, x represents any amino acid residue, and indices numbers indicate the respective number of consecutive amino acid residues. The abbreviation for the individual amino acid residues have their standard IUPAC meaning as defined above. A Clustal multiple alignment of the characteristic active site from various D-amino acids is shown in FIG. 5. Further potential DAAO enzymes comprising said motif are described in table below:

TABLE 1
Suitable D-amino acid oxidases from various organism. Acc.-No.
refers to protein sequence from SwisProt database.

| Acc. -No. | Gene Name | Description | Source Organism | Length |
|---|---|---|---|---|
| Q19564 | F18E3.7 | Putative D-amino acid | Caenorhabditis elegans | 334 |
| | | oxidase (EC 1.4.3.3) | | |
| | | (DAMOX) (DAO) | | |
| | | (DAAO) | | |
| P24552 | | D-amino acid oxidase | Fusarium solani | 361 |
| | | (EC 1.4.3.3) (DAMOX) | (subsp. pisi) | |
| | | (DAO) (DAAO) | (Nectria haematococca) | |
| P14920 | DAO, DAMOX | D-amino acid oxidase | Homo sapiens (Human) | 347 |
| | | (EC 1.4.3.3) (DAMOX) | | |
| | | (DAO) (DAAO) | | |
| P18894 | DAO, DAO1 | D-amino acid oxidase | Mus musculus (Mouse) | 346 |
| | | (EC 1.4.3.3) (DAMOX) | | |
| | | (DAO) (DAAO) | | |
| P00371 | DAO | D-amino acid oxidase | Sus scrofa (Pig) | 347 |
| | | (EC 1.4.3.3) (DAMOX) | | |
| | | (DAO) (DAAO) | | |
| P22942 | DAO | D-amino acid oxidase | Oryctolagus cuniculus | 347 |
| | | (EC 1.4.3.3) (DAMOX) | (Rabbit) | |
| | | (DAO) (DAAO) | | |
| O35078 | DAO | D-amino acid oxidase | Rattus norvegicus (Rat) | 346 |
| | | (EC 1.4.3.3) (DAMOX) | | |
| | | (DAO) (DAAO) | | |
| P80324 | DAO1 | D-amino acid oxidase | Rhodosporidium toruloides | 368 |
| | | (EC 1.4.3.3) (DAMOX) | (Yeast) | |
| | | (DAO) (DAAO) | (Rhodotorula gracilis) | |
| U60066 | DAO | D-amino acid oxidase | Rhodosporidium toruloides, | 368 |
| | | (EC 1.4.3.3) (DAMOX) | strain TCC 26217 | |
| | | (DAO) (DAAO) | | |
| Q99042 | DAO1 | D-amino acid oxidase | Trigonopsis variabilis | 356 |
| | | (EC 1.4.3.3) (DAMOX) | (Yeast) | |
| | | (DAO) (DAAO) | | |
| P31228 | DDO | D-aspartate oxidase (EC | Bos taurus (Bovine) | 341 |
| | | 1.4.3.1) (DASOX) (DDO) | | |
| Q99489 | DDO | D-aspartate oxidase (EC | Homo sapiens (Human) | 341 |
| | | 1.4.3.1) (DASOX) (DDO) | | |
| Q9C1L2 | NCU06558.1 | (AF309689) putative D- | Neurospora crassa | 362 |
| | | amino acid oxidase | | |
| | | G6G8.6 (Hypothetical | | |
| | | protein) | | |
| Q7SFW4 | NCU03131.1 | Hypothetical protein | Neurospora crassa | 390 |
| Q8N552 | | Similar to D-aspartate | Homo sapiens (Human) | 369 |
| | | oxidase | | |
| Q7Z312 | DKFZP686F04272 | Hypothetical protein | Homo sapiens (Human) | 330 |
| | | DKFZp686F04272 | | |
| Q9VM80 | CG11236 | CG11236 protein | Drosophila melanogaster | 341 |
| | | (GH12548p) | (Fruit fly) | |
| O01739 | F20H11.5 | F20H11.5 protein | Caenorhabditis elegans | 383 |
| O45307 | C47A10.5 | C47A10.5 protein | Caenorhabditis elegans | 343 |
| Q8SZN5 | CG12338 | RE73481p | Drosophila melanogaster | 335 |
| | | | (Fruit fly) | |
| Q9V5P1 | CG12338 | CG12338 protein | Drosophila melanogaster | 335 |
| | | (RE49860p) | (Fruit fly) | |
| Q86JV2 | | Similar to Bos taurus | Dictyostelium discoideum | 599 |
| | | (Bovine). D-aspartate | (Slime mold) | |
| | | oxidase (EC 1.4.3.1) | | |
| | | (DASOX) (DDO) | | |
| Q95XG9 | Y69A2AR.5 | Hypothetical protein | Caenorhabditis elegans | 322 |
| Q7Q7G4 | AGCG53627 | AgCP5709 (Fragment) | Anopheles gambiae | 344 |
| | | | str. PEST | |
| Q7PWY8 | AGCG53442 | AgCP12432 (Fragment) | Anopheles gambiae | 355 |
| | | | str. PEST | |
| Q7PWX4 | AGCG45272 | AgCP12797 (Fragment) | Anopheles gambiae | 373 |
| | | | str. PEST | |
| Q8PG95 | XAC3721 | D-amino acid oxidase | Xanthomonas axonopodis | 404 |
| | | | (pv. citri) | |
| Q8P4M9 | XCC3678 | D-amino acid oxidase | Xanthomonas campestris | 405 |
| | | | (pv. campestris) | |
| Q9X7P6 | SCO6740, | Putative D-amino acid | Streptomyces coelicolor | 320 |
| | SC5F2A.23C | oxidase | | |
| Q82MI8 | DAO, SAV1672 | Putative D-amino acid | Streptomyces avermitilis | 317 |
| | | oxidase | | |
| Q8VCW7 | DAO1 | D-amino acid oxidase | Mus musculus (Mouse) | 345 |
| Q9Z302 | | D-amino acid oxidase | Cricetulus griseus | 346 |
| | | | (Chinese hamster) | |
| Q9Z1M5 | | D-amino acid oxidase | Cavia porcellus | 347 |
| | | | (Guinea pig) | |
| Q922ZO | | Similar to D-aspartate | Mus musculus (Mouse) | 341 |
| | | oxidase | | |
| Q8R2R2 | | Hypothetical protein | Mus musculus (Mouse) | 341 |
| P31228 | | D-aspartate oxidase | B. taurus | 341 |

D-Amino acid oxidase (EC-number 1.4.3.3) can be isolated from various organisms, including but not limited to pig, human, rat, yeast, bacteria or fungi. Example organisms are Candida tropicalis, Trigonopsis variabilis, Neurospora crassa, Chlorella vulgaris, and Rhodotorula gracilis. A suitable D-amino acid metabolising polypeptide may be an eukaryotic enzyme, for example from a yeast (e.g. Rhodotorula gracilis), fungus, or animal or it may be a prokaryotic enzyme, for example, from a bacterium such as Escherichia coli. Examples of suitable polypeptides which metabolise D-amino acids are shown in Table 1 and Table 2.

TABLE 2
Suitable D-amino acid oxidases from various organism. Acc.-No.
refers to protein sequence from SwisProt database.

| | |
|---|---|
| Q19564 | Caenorhabditis elegans. F18E3.7. |
| P24552 | Fusarii solani (subsp. pisi) (Nectria haematococca). |
| JX0152 | Fusarium solani |
| P14920 | Homo sapiens (Human) |
| P18894 | Mus musculus (mouse) |
| P00371 | Sus scrofa (pig) |
| P22942 | Oryctolagus cuniculus (Rabbit) |
| O35078 | Rattus norvegicus (Rat) |
| P80324 | Rhodosporidium toruloides (Yeast) (Rhodotorula gracilis) |
| Q99042 | Trigonopsis variabilis |
| Q9Y7N4 | Schizosaccharomyces pombe (Fission yeast) SPCC1450 |
| O01739 | Caenorhabditis elegans.F20H11.5 |
| Q28382 | Sus scrofa (Pig). |
| O33145 | Mycobacterium leprae |
| Q9X7P6 | Streptomyces coelicolor.SCSF2A.23C |
| Q9JXF8 | Neisseria meningitidis (serogroup B). |
| Q9Z302 | Cricetulus griseus (Chinese hamster) |
| Q921M5 | D-AMINO ACID OXIDASE. Cavia parcellus (Guinea pig) |

Preferably the D-amino acid oxidase is selected from the enzymes encoded by a nucleic acid sequence or a corresponding amino acid sequences selected from

TABLE 3
Suitable D-amino acid oxidases from various organism. Acc.-No.
refers to protein sequence from GenBank database.

| GenBanc | | |
|---|---|---|
| Acc.-No | Organism | SEQ ID |
| U60066 | Rhodosporidium toruloides (Yeast) | SEQ ID NO: 1, 2 |
| Z71657 | Rhodotorula gracilis | |
| A56901 | Rhodotorula gracilis | |
| AF003339 | Rhodosporidium toruloides | |
| AF003340 | Rhodosporidium toruloides | |
| U53139 | Caenorhabditis elegans | SEQ ID NO: 3, 4 |
| D00809 | Nectria haematococca | SEQ ID NO: 5, 6 |
| Z50019. | Trigonopsis variabilis | SEQ ID NO: 7, 8 |
| NC_003421 | Schizosaccharomyces pombe | SEQ ID NO: 9, 10 |
| | (fission yeast) | |
| AL939129. | Streptomyces coelicolor A3(2) | SEQ ID NO: 11, 12 |
| AB042032 | Candida boidinii | SEQ ID NO: 13, 14 |

DAAO is a well-characterized enzyme, and both its crystal structure and its catalytic mechanism have been determined by high-resolution X-ray spectroscopy (Umhau S. et al. (2000) Proc. Natl. Acad. Sci. USA 97, 12463-12468). It is a flavoenzyme located in the peroxisome, and its recognized function in animals is detoxification of D-amino acids (Pilone M S (2000) Cell. Mol. Life. Sci. 57, 1732-174). In addition, it enables yeasts to use D-amino acids for growth (Yurimoto H et al. (2000) Yeast 16, 1217-1227). As demonstrated above, DAAO from several different species have been characterized and shown to differ slightly in substrate affinities (Gabler M et al. (2000) Enzyme Microb. Techno. 27, 605-611), but in general they display broad substrate specificity, oxidatively deaminating all D-amino acids (except D-glutamate and D-aspartate for EC 1.4.3.3. class DAAO enzymes; Pilone M S (2000) Cell. Mol. Life. Sci. 57,1732-174).

DAAO activity is found in many eukaryotes (Pilone M S (2000) Cell. Mol. Life. Sci. 57, 1732-174), but there is no report of DAAO activity in plants. The low capacity for D-amino acid metabolism in plants has major consequences for the way plants respond to D-amino acids. For instance, the results provided herein demonstrate that growth of A. thaliana in response to D-serine and/or D-alanine is inhibited even at quite low concentrations (FIG. 4a,b). On the other hand, some D-amino acids, like D-valine and D-isoleucine, have minor effects on plant growth (FIG. 4c,d) per se, but can be converted into toxic metabolites by action of a DAAO.

In a preferred embodiment D-amino acid oxidase expressed form the DNA-construct of the invention has preferably enzymatic activity against at least one of the amino acids selected from the group consisting of D-alanine, D-serine, D-isoleucine, D-valine, and derivatives thereof. Preferably said D-amino acid oxidase is selected from the group of amino acid sequences comprising

a) the sequences described by SEQ ID NO: 2, 4, 6, 8, 10, 12, and 14, and

b) the sequences having a sequence homology of at least 40%, preferably 60%, more preferably 80%, most preferably 95% with a sequence as described by SEQ ID NO: 2, 4,6, 8, 10, 12, and 14, and

c) the sequences hybridizing under low or high stringency conditions—preferably under high stringency conditions—with a sequence as described by SEQ ID NO: 2, 4, 6, 8, 10, 12, and 14.

Suitable D-amino acid oxidases also include fragments, mutants, derivatives, variants and alleles of the polypeptides exemplified above. Suitable fragments, mutants, derivatives, variants and alleles are those which retain the functional characteristics of the D-amino acid oxidase as defined above. Changes to a sequence, to produce a mutant, variant or derivative, may be by one or more of addition, insertion, deletion or substitution of one or more nucleotides in the nucleic acid, leading to the addition, insertion, deletion or substitution of one or more amino acids in the encoded polypeptide. Of course, changes to the nucleic acid that make no difference to the encoded amino acid sequence are included.

The D-amino acid oxidase of the invention may be expressed in the cytosol, peroxisome, or other intracellular compartment of the plant cell. Compartmentalisation of the D-amino acid metabolising polypeptide may be achieved by fusing the nucleic acid sequence encoding the DAAO polypeptide to a sequence encoding a transit peptide to generate a fusion protein. Gene products expressed without such transit peptides generally accumulate in the cytosol. The localization of expressed DAAO in the peroxisome produces H₂O₂ that can be metabolised by the H₂O₂ degrading enzyme catalase. Higher levels of D-amino acids may therefore be required to produce damaging levels of H₂O₂. Expression of DAAO in the cytosol, where levels of catalase activity are lower, reduces the amount of D-amino acid required to produce damaging levels H₂O₂. Expression of DAAO in the cytosol may be achieved by removing peroxisome targeting signals or transit peptides from the encoding nucleic acid sequence. For example, the dao1 gene (EC: 1.4.3.3: GenBank Acc.-No.: U60066) from the yeast Rhodotorula gracilis (Rhodosporidium toruloides) was cloned as described (WO 03/060133). The last nine nucleotides encode the signal peptide SKL, which guides the protein to the peroxisome sub-cellular organelle. Although no significant differences were observed between cytosolic, and peroxisomal expressed DAAO, the peroxisomal construction was found to be marginally more effective than the cytosolic version in respect of inhibiting the germination of the DAAO transgenic plants on 30 mM D-Asn. However, both constructs are inhibited significantly more than the wild-type and may thus be used for conditional counter-selection.

In another preferred embodiment the (non-phytotoxic, but metabolizable into phytotoxic) compound M is preferably comprising a D-amino acid structure selected from the group consisting of D-isoleucine, D-valine, D-asparagine, D-leucine, D-lysine, D-proline, and D-glutamine, and derivatives thereof. Preferably, M is comprising and/or consisting of D-isoleucine, D-valine, or derivatives thereof.

There are multiple D-amino acid oxidases known in the art which may be employed within the method of the invention. For example the D-amino acid oxidase is described by a sequence of the group consisting of sequences described by GenBank or SwisProt Acc. No. JX0152, O01739, O33145, O35078, O45307, P00371, P14920, P18894, P22942, P24552, P31228, P80324, Q19564, Q28382, Q7PWX4, Q7PWY8, Q7Q7G4, Q7SFW4, Q7Z312, Q82MI8, Q86JV2, Q8N552, Q8P4M9, Q8PG95, Q8R2R2, Q8SZN5, Q8VCW7, Q921M5, Q922Z0, Q95XG9, Q99042, Q99489, Q9C1L2, Q9JXF8, Q9V5PI, Q9VM80, Q9X7P6, Q9Y7N4, Q9Z1M5, Q9Z302, and U60066. Preferably, the D-amino acid oxidase is selected from the group of amino acid sequences consisting of

a) the sequences described by SEQ ID NO: 2, 4, 6, 8, 10, 12, and 14, and

b) the sequences having a sequence homology of at least 40%, preferably 60%, more preferably 80%, most preferably 95% with a sequence as described by SEQ ID NO: 2, 4, 6, 8, 10, 12, and 14, and

c) the sequences hybridizing under low or high stringency conditions—preferably under high stringency conditions—with a sequence as described by SEQ ID NO: 2, 4, 6, 8, 10, 12, and 14.

Another embodiment of the invention is related to selective herbicidal composition comprising at least one compound M, wherein M is comprising a D-amino acid structure, preferably selected from the group consisting of D-isoleucine, D-valine, D-asparagine, D-leucine, D-lysine, D-proline, and D-glutamine, and derivatives thereof. In a preferred embodiment the selective herbicidal composition comprising at least one compound selected from the group consisting of D-isoleucine, D-valine, and derivatives thereof. An other embodiment of the invention is related to the use of a selective herbicidal composition of the invention to prevent or suppress unwanted growth of transgenic plants.

The term “combination” or “combined” with respect to the relation between the first and the second expression cassette is to be understood in the broad sense and is intended to mean any mode operation which is linking the functionality of the two expression cassettes. The first and the second expression cassette may be comprised in one DNA construct but may also be separate molecules.

### II. Compound M and the Selective Herbicidal Composition

The term “Compound M” means one or more chemical substances (i.e. one chemical compound or a mixture of two or more compounds) which is non-phytotoxic or moderately phytotoxic against plant cells not functionally expressing said D-amino acid oxidase, and which can be metabolized by said D-amino acid oxidase into one or more compound(s) N which are phytotoxic or more phytotoxic than compound M.

The term “phytotoxic”, “phytotoxicity” or “phytotoxic effect” as used herein is intended to mean any measurable, negative effect on the physiology of a plant or plant cell resulting in symptoms including (but not limited to) for example reduced or impaired growth, reduced or impaired photosynthesis, reduced or impaired cell division, reduced or impaired regeneration (e.g., of a mature plant from a cell culture, callus, or shoot etc.), reduced or impaired fertility etc. Phytotoxicity may further include effects like e.g., necrosis or apoptosis. In a preferred embodiment results in an reduction of growth or regenerability of at least 50%, preferably at least 80%, more preferably at least 90% in comparison with a plant which was not treated with said phytotoxic compound.

The term “non-phytotoxic” means that no statistically significant difference in physiology can be observed between plant cells or plants (not comprising a functional D-amino acid oxidase) and the same plant cells or plants treated with compound M or untreated plants.

The term “moderate phytotoxic” means a reduction of a physiological indicator (as exemplified above like e.g., growth or regenerability) for treated plant cells or plants—not comprising a functional D-amino acid oxidase—in comparison with untreated plants or plant cells (regardless whether expressing said D-amino acid oxidase or not ) not irreversibly effecting growth and/or performance of said treated plants or plant cells (but using the compound in a concentration sufficient to allow for distinguishing and/or separating transgenic plants (i.e., comprising said dual function marker) from non-transgenic plants (i.e., not comprising said marker)). Preferably, the reduction of a physiological indicator for said treated plant cells is not more then 30%, preferably not more then 15%, more preferably not more then 10%.

The phytotoxic compound M is metabolized by said D-amino acid oxidase into one or more compound(s) N which are phytotoxic or more phytotoxic than compound M. In an improved embodiment the toxicity (as for example assessed by one of the physiological indicators exemplified above like e.g., growth or regenerability) of the compound M is increased in a way that one or more physiological indicator (as exemplified above like e.g., growth or regenerability) are reduced by at least 20%, preferably at least 40%, more preferably at least 60%, most preferably at least 90%. The phytotoxic effect of compound N in comparison to compound M is increased by at least 100% (i.e. twice), preferably at least 500% (i.e. 5-times), more preferably at least 1000% (i.e. 10 times).

Various chemical compounds and mixtures thereof can be used as compound M. The person skilled in the art is aware of assay systems to assess the phytotoxicity of these compounds and the capability of a D-amino oxidase to metabolize said compounds in a way described above leading to increased phytotoxicity.

Preferably at least one of the chemical substances comprised in compound M comprises a D-amino acid structure.

As used herein the term a “D-amino acid structure” (such as a “D-leucine structure”, a “D-phenylalanine structure” or a “D-valine structure”) is intended to include the D-amino acid, as well as analogues, derivatives and mimetics of the D-amino acid that maintain the functional activity of the compound (discussed further below). For example, the term “D-phenylaianine structure” is intended to include D-phenylalanine as well as D-pyridylalanine and D-homophenylalanine. The term “D-leucine structure” is intended to include D-leucine, as well as substitution with D-valine or other natural or non-natural amino acid having an aliphatic side chain, such as D-norleucine. The term “D-valine structure” is intended to include D-valine, as well as substitution with D-leucine or other natural or non-natural amino acid having an aliphatic side chain.

The D-amino acid employed may be modified by an amino-terminal or an carboxy-terminal modifying group. The amino-terminal modifying group may be—for example—selected from the group consisting of phenylacetyl, diphenylacetyl, triphenylacetyl, butanoyl, isobutanoyl hexanoyl, propionyl, 3-hydroxybutanoyl, 4-hydroxybutanoyl, 3-hydroxypropionoyl, 2,4-dihydroxybutyroyl, 1-Adamantanecarbonyl, 4-methylvaleryl, 2-hydroxyphenylacetyl, 3-hydroxyphenylacetyl, 4-hydroxyphenylacetyl, 3,5-dihydroxy-2-naphthoyl, 3,7-dihydroxy-2-napthoyl, 2-hydroxycinnamoyl, 3-hydroxycinnamoyl, 4-hydroxycinnamoyl, hydrocinnamoyl, 4-formylcinnamoyl, 3-hydroxy-4-methoxycinnamoyl, 4-hydroxy-3-methoxycinnamoyl, 2-carboxycinnamoyl, 3,4,-dihydroxyhydrocinnamoyl, 3,4-dihydroxycinnamoyl, trans-Cinnamoyl, (.+−.)-mandelyl. (.+−.)-mandelyl-(.+−.)-mandelyl, glycolyl, 3-formylbenzoyl, 4-formylbenzoyl, 2-formylphenoxyacetyl, 8-formyl-1-napthoyl, 4-(hydroxymethyl)benzoyl, 3-hydroxybenzoyl, 4-hydroxybenzoyl, 5-hydantoinacetyl, L-hydroorotyl, 2,4-dihydroxybenzoyl, 3-benzoylpropanoyl, (.+−.)-2,4-dihydroxy-3,3-dimethylbutanoyl, DL-3-(4-hydroxyphenyl)lactyl, 3-(2-hydroxyphenyl)propionyl, 4-(2-hydroxyphenyl)propionyl, D-3-phenyllactyl, 3-(4-hydroxyphenyl)propionyl, L-3-phenyllactyl, 3-pyridylacetyl, 4-pyridylacetyl, isonicotinoyl, 4-quinolinecarboxyl, 1-isoquinolinecarboxyl and 3-isoquinolinecarboxyl. The carboxy-terminal modifying group may be—for example—selected from the group consisting of an amide group, an alkyl amide group, an aryl amide group and a hydroxy group.

The terms “analogue”, “derivative” and “mimetic” as used herein are intended to include molecules which mimic the chemical structure of a D-amino acid structure and retain the functional properties of the D-amino acid structure. Approaches to designing amino acid or peptide analogs, derivatives and mimetics are known in the art. For example, see Farmer, P. S. in Drug Design (E. J. Ariens, ed.) Academic Press, N.Y., 1980, vol. 10, pp. 119-143; Ball. J. B. and Alewood, P. F. (1990) J. Mol. Recognition 3:55; Morgan, B. A. and Gainor, J. A. (1989) Ann. Rep. Med. Chem. 24:243; and Freidinger, R. M. (1989) Trends Pharmacol. Sci. 10:270. See also Sawyer, T. K. (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith, A. B. 3rd, et al. (1995) J. Am. Chem. Soc. 117:11113-11123; Smith, A. B. 3rd, et al. (1994) J. Am. Chem. Soc. 116:9947-9962; and Hirschman, R., et al. (1993) J. Am. Chem. Soc. 115:12550-12568.

As used herein, a “derivative” of a compound M (e.g., a D-amino acid) refers to a form of M in which one or more reaction groups on the compound have been derivatized with a substituent group. Examples of peptide derivatives include peptides in which an amino acid side chain, or the amino- or carboxy-terminus has been derivatized. As used herein an “analogue” of a compound M refers to a compound which retains chemical structures of M necessary for functional activity of M yet which also contains certain chemical structures which differ from M, respectively. As used herein, a “mimetic” of a compound M refers to a compound in which chemical structures of M necessary for functional activity of M have been replaced with other chemical structures which mimic the conformation of M, respectively.

Analogues are intended to include compounds in which one or more D-amino acids are substituted with a homologous amino acid such that the properties of the original compound are maintained. Preferably conservative amino acid substitutions are made at one or more amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Non-limiting examples of homologous substitutions that can be made include substitution of D-phenylalanine with D-tyrosine, D-pyridylalanine or D-homophenylalanine, substitution of D-leucine with D-valine or other natural or non-natural amino acid having an aliphatic side chain and/or substitution of D-valine with D-leucine or other natural or non-natural amino acid having an aliphatic side chain.

Other possible modifications include N-alkyl (or aryl) substitution, or backbone crosslinking to construct lactams and other cyclic structures. Other derivatives include C-terminal hydroxymethyl derivatives, 0-modified derivatives (e.g., C-terminal hydroxymethyl ides such as alkylamides and hydrazides.

In certain embodiments the D-amino acid structure is coupled directly or indirectly to at least one modifying group (abbreviated as MG). The term “modifying group” is intended to include structures that are directly attached to the D-amino acid structure (e.g., by covalent coupling), as well as those that are indirectly attached (e.g., by a stable non-covalent association or by covalent coupling to additional amino acid residues). For example, the modifying group can be coupled to the amino-terminus or carboxy-terminus of a D-amino acid structure. Modifying groups covalently coupled to the D-amino acid structure can be attached by means and using methods well known in the art for linking chemical structures, including, for example, amide, alkylamino, carbamate, urea or ester bonds. In a preferred embodiment, the modifying group(s) comprises a cyclic, heterocyclic, polycyclic or branched alkyl group.

No endogenous D-amino acid oxidase activity has been reported in plants. Compound M, respectively, as substrates for the D-amino acid oxidase may be a D-amino acid structure comprising the structure of D-arginine, D-glutamate, D-alanine, D-aspartate, D-cysteine, D-glutamine, D-histidine, D-isoleucine, D-leucine, D-lysine, D-methionine, D-asparagine, D-phenylalanine, D-proline, D-serine, D-threonine, D-tryptophane, D-tyrpsine or D-valine. Preferably compound M is comprising D-arginine, D-glutamate, D-alanine, D-aspartate, D-cysteine, D-glutamine, D-histidine, D-isoleucine, D-leucine, D-lysine, D-methionine, D-asparagine, D-phenylalanine, D-proline, D-serine, D-threonine, D-tryptophane, D-tyrosine or D-valine. Other suitable substrates for D-amino acid metabolising enzymes include non-protein dextrorotatory amino acids, precursors of dextrorotatory amino acids and dextrorotatory amino acid derivatives. Suitable precursors include D-ornithine and D-citrulline.

Preferably compound M is comprising a substance comprising E structure selected from the group of consisting of D-isoleucine, D-valine, D-asparagine, D-leucine, D-lysine, D-proline, and D-glutamine, more preferably a structure selected from the group consisting of D-isoleucine, and D-valine. Most preferably compound M is comprising a substance comprising the structure of D-isoleucine.

Preferably compound M is comprising a substance selected from the group of consisting of D-isoleucine, D-valine, D-asparagine, D-leucine, D-lysine, D-proline, and D-glutamine, more preferably selected from the group consisting of D-isoleucine, and D-valine. Most preferably compound M is comprising D-isoleucine.

The fact that compound M preferably comprise a D-amino acid structure does not rule out the presence of L-amino acid structures or L-amino acids. For some applications it may be preferred (e.g., for cost reasons) to apply a racemic mixture of D- and L-amino acids (or a mixture with enriched content of D-amino acids). Preferably, the ratio of the D-amino acid to the corresponding L-enantiomer is at least 1:1, preferably 2:1, more preferably 5:1, most preferably 10:1 or 100:1.

The preferred compound may be used in isolated form or in combination with other substances.

The term “herbicidal composition” or “selective herbicidal” composition as used herein is preferably intended to mean any composition comprising at least one compound M (as defined above) and at least one adjuvant facilitating application of the composition as a herbicide. For the purpose of application, the compound M is advantageously used together with the adjuvants conventionally employed in the art of formulation, and are therefore formulated in known manner, e.g. into emulsifiable concentrates, coatable pastes, directly sprayable or dilutable solutions, dilute emulsions, wettable powders, soluble powders, dusts, granulates, and also encapsulations in e.g. polymer substances. As with the nature of the compositions to be used, the methods of application, such as spraying, atomising, dusting, scattering, coating or pouring, are chosen in accordance with the intended objectives and the prevailing circumstances.

The formulations, i.e. the compositions, preparations or mixtures containing compound M (active ingredient), and, where appropriate, a solid or liquid adjuvant, are prepared in known manner, e.g. by homogeneously mixing and/or grinding the active ingredients with extenders, e.g. solvents, solid carriers and, where appropriate, surface-active compounds (surfactants).

Suitable solvents are: aromatic hydrocarbons, preferably the fractions containing 8 to 12 carbon atoms, e.g. xylene mixtures or substituted naphthalenes, phthalates such as dibutyl phthalate or dioctyl phthalate, aliphatic hydrocarbons such as cyclohexane or paraffins, alcohols and glycols and their ethers and esters, such as ethanol, ethylene glycol, ethylene glycol monomethyl or monoethyl ether, ketones such as cyclohexanone, strongly polar solvents such as N-methyl-2-pyrrolidone, dimethyl sulfoxide or dimethylformamide, as well as vegetable oils or epoxidised vegetable oils, such as epoxidised coconut oil or soybean oil; or—preferably—water.

The solid carriers used e.g. for dusts and dispersible powders are normally natural mineral fillers such as calcite, talcum, kaolin, montmorillonite or attapulgite. In order to improve the physical properties it is also possible to add highly dispersed silicic acid or highly dispersed absorbent polymers. Suitable granulated adsorptive carriers are porous types, for example pumice, broken brick, sepiolite or bentonite; and suitable non-sorbent carriers are, for example, calcite or sand. In addition, a great number of pregranulated materials of inorganic or organic nature can be used, e.g. especially dolomite or pulverised plant residues.

Depending on the nature of the compound M to be formulated suitable surface-active compounds are nonionic, cationic and/or anionic surfactants having good emulsifying, dispersing and wetting properties. The term “surfactants” will also be understood as comprising mixtures of surfactants.

Both so-called water-soluble soaps and also water-soluble synthetic surface-active compounds are suitable anionic surfactants. Suitable soaps are the alkali metal salts, alkaline earth metal salts or unsubstituted or substituted ammonium salts of higher fatty acids (C₁₀-C₂₂), e.g. the sodium or potassium salts of oleic or stearic acid or of natural fatty acid mixtures which can be obtained e.g. from coconut oil or tallow oil. Fatty acid methyltaurin salts may also be mentioned as surfactants.

More frequently, however, so-called synthetic surfactants are used, especially fatty sulfonates, fatty sulfates, sulfonated benzimidazole derivatives or alkylarylsulfonates. The fatty sulfonates or sulfates are usually in the form of alkali metal salts, alkaline earth metal salts or unsubstituted or substituted ammonium salts and contain a C.sub.8-C.sub.22 alkyl radical which also includes the alkyl moiety of acyl radicals, e.g. the sodium or calcium salt of lignosulfonic acid, of dodecylsulfate or of a mixture of fatty alcohol sulfates obtained from natural fatty acids. These compounds also comprise the salts of sulfated and sulfonated fatty alcohol/ethylene oxide adducts. The sulfonated benzimidazole derivatives preferably contain 2 sulfonic acid groups and one fatty acid radical containing 8 to 22 carbon atoms. Examples of alkylarylsulfonates are the sodium, calcium or triethanolamine salts of dodecylbenzenesulfonic acid, dibutylnaphthalenesulfonic acid, or of a condensate of naphthalenesulfonic acid and formaldehyde. Also suitable are corresponding phosphates, e.g. salts of the phosphoric acid ester of an adduct of p-nonylphenol with 4 to 14 moles of ethylene oxide, or phospholipids.

Non-ionic surfactants are preferably polyglycol ether derivatives of aliphatic or cycloaliphatic alcohols, saturated or unsaturated fatty acids and alkylphenols, said derivatives contains 3 to 30 glycol ether groups and 8 to 20 carbon atoms in the (aliphatic) hydrocarbon moiety and 6 to 18 carbon atoms in the alkyl moiety of the alkylphenols. Further suitable non-ionic surfactants are the water-soluble adducts of polyethylene oxide with polypropylene glycol, ethylenediaminopolypropylene glycol and alkylpolypropylene glycol containing 1 to 10 carbon atoms in the alkyl chain, which adducts contain 20 to 250 ethylene glycol ether groups and 10 to 100 propylene glycol ether groups. These compounds usually contain 1 to 5 ethylene glycol units per propylene glycol unit. Representative examples of non-ionic surfactants are nonylphenolpolyethoxyethanols, castor oil polyglycol ethers, polypropylene/polyethylene oxide adducts, tributylphenoxy-polyethoxyethanol, polyethylene glycol and octylphenoxypolyethoxyethanol. Fatty acid esters of polyoxyethylene sorbitan, e.g. polyoxyethylene sorbitan trioleate, are also suitable.

Cationic surfactants are preferably quaternary ammonium salts which contain, as N-substituent, at least one C₈ -C₂₂ alkyl radical and, as further substituents, unsubstituted or halogenated lower alkyl, benzyl or hydroxy-lower alkyl radicals. The salts are preferably in the form of halides, methylsulfates or ethylsulfates, e.g. stearyltrimethylammonium chloride or benzyldi(2-chloroethyl)ethylammonium bromide.

The surfactants customarily employed in the art of formulation are described e.g. in the following publications: “McCutcheon's Detergents and Emulsifiers Annual” M C Publishing Corp., Ridgewood, N.J., 1981. Stache, H., “Tensid-Taschenbuch”, Carl Hanser Verlag Munich/Vienna 1981.

The compositions usually contain 0.1 to 99% by weight, preferably 0.1 to 95% by weight, of a compound X or M, 1 to 99.9% by weight, preferably 5 to 99.8% by weight, of a solid or liquid adjuvant and 0 to 25% by weight, preferably 0.1 to 25% by weight, of a surfactant.

The compositions may also contain further ingredients such as stabilizers, antifoams, viscosity regulators, binders, tackifiers as well as fertilizers or other active ingredients for obtaining special effects.

Various methods and techniques are suitable for employing compound X or M or compositions containing them for treating plant cells or plants. Such method may include

i) Incorporation into liquid or solidified media or substrates utilized during transformation, regeneration or growth of plant cells, plant material or plants.

ii) Seed dressing

iii) Application by spraying (e.g. from a tank mixture utilizing a liquid formulation)

Suitable concentrations of the active ingredient M (e.g., preferably D-isoleucine) in the herbicidal composition of the invention are preferably in the range of 0.3 to 100 mM, more preferably 1 mM to 80 mM, most preferably 5 mM to 50 mM.

Ill. The DNA Constructs of the Invention

A transgenic expression cassette for a D-amino acid oxidase suitable for carrying out the invention may comprise a sequence encoding said D-amino acid oxidase (as defined above) operably linked to a promoter functional in plants. Various promoters functional in plants are known in the art (see above). Preferably for the present invention the promoter is a constitutive promoter allowing for expression of the D-amino oxidase in all or substantially all tissues and during most of the developmental stages. Examples for said constitutive promoters are given above. However other promoters (e.g., with activity in green tissues like leaves) may be useful. Further preferred constitutive promoters are the nitrilase promoter from Arabidopsis thaliana (WO 03/008596) and the Pisum sativum ptxA promoter (e.g., as incorporated in the construct described by SEQ ID NO: 16; base pair 1866-2728, complementary orientation).

The DNA construct may—beside a promoter sequence—comprise additional genetic control sequences. The term “genetic control sequences” is to be understood in the broad sense and refers to all those sequences which affect the making or function of the DNA construct to the invention or an expression cassette comprised therein. Preferably, an expression cassettes according to the invention encompass 5′-upstream of the respective nucleic acid sequence to be expressed a promoter and 3′-downstream a terminator sequence as additional genetic control sequence, and, if appropriate, further customary regulatory elements, in each case in operable linkage with the nucleic acid sequence to be expressed.

Genetic control sequences are described, for example, in “Goeddel; Gene Expression Technology: Methods in Enzymology 185, Academic Press, San Diego, Calif. (1990)” or “Gruber and Crosby, in: Methods in Plant Molecular Biology and Biotechnolgy, CRC Press, Boca Raton, Fla., eds.: Glick and Thompson, Chapter 7, 89-108” and the references cited therein.

Examples of such control sequences are sequences to which inductors or repressors bind and thus regulate the expression of the nucleic acid. Genetic control sequences furthermore also encompass the 5′-untranslated region, introns or the non-coding 3′-region of genes. It has been demonstrated that they may play a significant role in the regulation of gene expression. Thus, it has been demonstrated that 5′-untranslated sequences are capable of enhancing the transient expression of heterologous genes. Furthermore, they may promote tissue specificity (Rouster J et al. (1998) Plant J 15:435-440.). Conversely, the 5′-untranslated region of the opaque-2 gene suppresses expression. Deletion of the region in question leads to an increased gene activity (Lohmer S et al. (1993) Plant Cell 5:65-73). Genetic control sequences may also encompass ribosome binding sequences for initiating translation. This is preferred in particular when the nucleic acid sequence to be expressed does not provide suitable sequences or when they are not compatible with the expression system.

The expression cassette can advantageously comprise one or more of what are known as enhancer sequences in operable linkage with the promoter, which enable the increased transgenic expression of the nucleic acid sequence. Additional advantageous sequences, such as further regulatory elements or terminators, may also be inserted at the 3′ end of the nucleic acid sequences to be expressed recombinantly. One or more copies of the nucleic acid sequences to be expressed recombinantly may be present in the gene construct. Genetic control sequences are furthermore understood as meaning sequences which encode fusion proteins consisting of a signal peptide sequence.

Polyadenylation signals which are suitable as genetic control sequences are plant polyadenylation signals, preferably those which correspond essentially to T-DNA polyadenylation signals from Agrobacterium tumefaciens. Examples of particularly suitable terminator sequences are the OCS (octopine synthase) terminator and the NOS (nopaline synthase) terminator.

The DNA-constructs of the invention may encompass further nucleic acid sequences. Such nucleic acid sequences may preferably constitute expression cassettes. Said further sequences may include but shall not be limited to:

i) Additional counter selection marker as described above. Or additional negative or positive selection marker. Negative selection markers are most often employed in methods for producing transgenic cells or organisms. Such negative selection markers confer for example a resistance to a biocidal compound such as a metabolic inhibitor (e.g., 2-deoxyglucose-6-phosphate, WO 98/45456), antibiotics (e.g., kanamycin, G 418, bleomycin or hygromycin) or herbicides (e.g., phosphinothricin or glyphosate). Examples—especially suitable for plant transformation—are:

Phosphinothricin acetyltransferases (PAT; also named Bialophos® resistance; bar; de Block 1987; EP 0 333 033; U.S. Pat. No. 4,975,374)

5-enolpyruvylshikimate-3-phosphate synthase (EPSPS) conferring resistance to Glyphosate® (N-(phosphonomethyl)glycine) (Shah 1986)

Glyphosate® degrading enzymes (Glyphosate® oxidoreductase; gox),

Dalapon® inactivating dehalogenases (deh)

sulfonylurea- and imidazolinone-inactivating acetolactate synthases (for example mutated ALS variants with, for example, the S4 and/or Hra mutation

Bromoxynil® degrading nitrilases (bxn)

Kanamycin- or G418- resistance genes (NPTII; NPTI) coding e.g., for neomycin phosphotransferases (Fraley 1983)

2-Desoxyglucose-6-phosphate phosphatase (DOGR1-Gene product; WO 98/45456; EP 0 807 836) conferring resistance against 2-desoxyglucose (Randez-Gil 1995).

hygromycin phosphotransferase (HPT), which mediates resistance to hygromycin (Vanden Elzen 1985).

dihydrofolate reductase (Eichholtz 1987)

D-amino acid metabolizing enzyme (e.g., D-amino acid dehydratases or oxidases; WO 03/060133)

Additional negative selectable marker genes of bacterial origin that confer resistance to antibiotics include the aadA gene, which confers resistance to the antibiotic spectinomycin, gentamycin acetyl transferase, streptomycin phosphotransferase (SPT), aminoglycoside-3-adenyl transferase and the bleomycin resistance determinant (Hayford 1988; Jones 1987; Svab 1990; Hille 1986).

Additional selection markers are those which do not result in detoxification of a biocidal compound but confer an advantage by increased or improved regeneration, growth, propagation, multiplication as the like of the cell or organism comprising such kind of “positive selection marker”. Examples are isopentenyltransferase (a key enzyme of the cytokinin biosynthesis facilitating regeneration of transformed plant cells by selection on cytokinin-free medium; Ebinuma 2000a; Ebinuma 2000b). Additional positive selection markers, which confer a growth advantage to a transformed plant cells in comparison with a non-transformed one, are described e.g., in EP-A 0 601 092. Growth stimulation selection markers may include (but shall not be limited to) β-Glucuronidase (in combination with e.g., a cytokinin glucuronide), mannose-6-phosphate isomerase (in combination with mannose), UDP-galactose-4-epimerase (in combination with e.g., galactose).

ii) Report genes which encode readily quantifiable proteins and which, via intrinsic color or enzyme activity, ensure the assessment of the transformation efficacy or of the location or timing of expression. Very especially preferred here are genes encoding reporter proteins (see also Schenborn E, Groskreutz D. Mol Biotechnol. 1999; 13(1):29-44) such as

“green fluorescence protein” (GFP,) (Chui W L et al., Curr Biol 1996, 6:325-330; Leffel S M et al., Biotechniques. 23(5):912-8, 1997; Sheen et al. (1995) Plant Journal 8(5):777-784; Haseloff et al. (1997) Proc Natl Acad Sci USA 94(6):2122-2127; Reichel et al.(1996) Proc. Natl Acad Sci USA 93(12):5888-5893; Tian et al. (1997) Plant Cell Rep 16:267-271; WO 97/41228).

Chloramphenicol transferase,

luciferase (Millar et al., Plant Mol Biol Rep 1992 10:324-414; Ow et al. (1986) Science, 234:856-859); permits the detection of bioluminescence,

β-galactosidase, encodes an enzyme for which a variety of chromogenic substrates are available,

β-glucuronidase (GUS) (Jefferson et al., EMBO J. 1987, 6, 3901-3907) or the uidA gene, which encodes an enzyme for a variety of chromogenic substrates,

R locus gene product: protein which regulates the production of anthocyanin pigments (red coloration) in plant tissue and thus makes possible the direct analysis of the promotor activity without the addition of additional adjuvants or chromogenic substrates (Dellaporta et al., In: Chromosome Structure and Function: Impact of New Concepts, 18th Stadler Genetics Symposium, 11:263-282, 1988),

β-lactamase (Sutcliffe (1978) Proc Natl Acad Sci USA 75:3737-3741), enzyme for a variety of chromogenic substrates (for example PADAC, a chromogenic cephalosporin),

xylE gene product (Zukowsky et al. (1983) Proc Natl Acad Sci USA 80:1101-1105), catechol dioxygenase capable of converting chromogenic catechols,

alpha-amylase (Ikuta et al. (1990) Bio/technol. 8:241-242),

tyrosinase (Katz et al.(1983) J Gene Microbiol 129:2703-2714), enzyme which oxidizes tyrosine to give DOPA and dopaquinone which subsequently form melanine, which is readily detectable,

aequorin (Prasher et al.(1985) Biochem Biophys Res Commun 126(3):1259-1268), can be used in the calcium-sensitive bioluminescence detection.

The DNA construct according to the invention and any vectors derived therefrom may comprise further functional elements. The term “further functional elements” is to be understood in the broad sense. It preferably refers to all those elements which affect the generation, multiplication, function, use or value of said DNA construct or vectors comprising said DNA construct, or cells or organisms comprising the before mentioned. These further functional elements may include but shall not be limited to:

i) Origins of replication which ensure replication of the expression cassettes or vectors according to the invention in, for example, E. coli. Examples which may be mentioned are ORI (origin of DNA replication), the pBR322 ori or the P15A ori (Sambrook et al.: Molecular Cloning. A Laboratory Manual, 2^{nd} ed. Cold Spring Harbor Laboratory Press, Cold Spring Harbor, N.Y., 1989).

ii) Multiple cloning sites (MCS) to enable and facilitate the insertion of one or more nucleic acid sequences.

iii) Sequences which make possible homologous recombination or insertion into the genome of a host organism.

iv) Elements, for example border sequences, which make possible the Agrobacterium-mediated transfer in plant cells for the transfer and integration into the plant genome, such as, for example, the right or left border of the T-DNA or the vir region.

### IV. Construction of the DNA Constructs of the Invention

Typically, constructs to be introduced into these cells are prepared using transgene expression techniques. Recombinant expression techniques involve the construction of recombinant nucleic acids and the expression of genes in transfected cells.

Molecular cloning techniques to achieve these ends are known in the art. A wide variety of cloning and in vitro amplification methods suitable for the construction of recombinant nucleic acids are well-known to persons of skill. Examples of these techniques and instructions sufficient to direct persons of skill through many cloning exercises are found in Berger and Kimmel, Guide to Molecular Cloning Techniques, Methods in Enzymology, Vol. 152, Academic Press, hic., San Diego, Calif. (Berger) ; T. Maniatis, E. F. Fritsch and J. Sambrook, Molecular Cloning: A Laboratory Manual, Cold Spring Harbor Laboratory, Cold Spring Harbor, N.Y. (1989), in T. J. Silhavy, M. L. Berman and L. W. Enquist, Experiments with Gene Fusions, Cold Spring Harbor Laboratory, Cold Spring Harbor, N.Y. (1984) and Current Protocols in Molecular Biology, F. M. Ausubel et al., eds., Current Protocols, a joint venture between Greene Publishing Associates, Inc. and John Wiley & Sons, Inc., (1998 Supplement). Preferably, the DNA construct according to the invention is generated by joining the abovementioned essential constituents of the DNA construct together in the abovementioned sequence using the recombination and cloning techniques with which the skilled worker is familiar.

Generally, a gene to be expressed will be present in an expression cassette, meaning that the gene is operably linked to expression control signals, e. g., promoters and terminators, that are functional in the host cell of interest. The genes that encode the sequence-specific DNA cleaving enzyme and, optionally, the selectable marker, will also be under the control of such signals that are functional in the host cell. Control of expression is most easily achieved by selection of a promoter. The transcription terminator is not generally as critical and a variety of known elements may be used so long as they are recognized by the cell. The invention contemplates polynucleotides operably linked to a promoter in the sense or antisense orientation.

A DNA construct of the invention (or an expression cassette or other nucleic acid employed herein) is preferably introduced into cells using vectors into which these constructs or cassettes are inserted. Examples of vectors may be plasmids, cosmids, phages, viruses, retroviruses or else agrobacteria.

The construction of polynucleotide constructs generally requires the use of vectors able to replicate in bacteria. A plethora of kits are commercially available for the purification of plasmids from bacteria. For their proper use, follow the manufacturer's instructions (see, for example, EasyPrep™, FlexiPrep™, both from Pharmacia Biotech; StrataClean™, from Stratagene; and, QIAexpress™ Expression System, Qiagen). The isolated and purified plasmids can then be further manipulated to produce other plasmids, used to transfect cells or incorporated into Agrobacterium tumefaciens to infect and transform plants. Where Agrobacterium is the means of transformation, shuttle vectors are constructed.

However, an expression cassette (e.g., for an excision enzyme) may also be constructed in such a way that the nucleic acid sequence to be expressed (for example one encoding a excision enzyme) is brought under the control of an endogenous genetic control element, for example a promoter, for example by means of homologous recombination or else by random insertion. Such constructs are likewise understood as being expression cassettes for the purposes of the invention. The skilled worker furthermore knows that nucleic acid molecules may also be expressed using artificial transcription factors of the zinc finger protein type (Beerli R R et al. (2000) Proc Natl Acad Sci USA 97(4):1495-500). These factors can be adapted to suit any sequence region and enable expression independently of certain promoter sequences.

### V. Target Organisms

The methods of the invention are useful for obtaining marker-free plants, or cells, parts, tissues, harvested material derived therefrom. Accordingly, another subject matter of the invention relates to transgenic plants or plant cells comprising in their genome, preferably in their nuclear, chromosomal DNA, the DNA construct according to the invention, and to cells, cell cultures, tissues, parts or propagation material—such as, for example, in the case of plant organisms leaves, roots, seeds, fruit, pollen and the like—derived from such plants.

The term “plant” includes whole plants, shoot vegetative organs/structures (e. g. leaves, stems and tubers), roots, flowers and floral organs/structures (e. g. bracts, sepals, petals, stamens, carpels, anthers and ovules), seeds (including embryo, endosperm, and seed coat) and fruits (the mature ovary), plant tissues (e. g. vascular tissue, ground tissue, and the like) and cells (e. g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

Included within the scope of the invention are all genera and species of higher and lower plants of the plant kingdom. Included are furthermore the mature plants, seed, shoots and seedlings, and parts, propagation material (for example seeds and fruit) and cultures, for example cell cultures, derived therefrom.

Preferred are plants and plant materials of the following plant families: Amaranthaceae, Brassicaceae, Carophyllaceae, Chenopodiaceae, Compositae, Cucurbitaceae, Labiatae, Leguminosae, Papilionoideae, Liliaceae, Linaceae, Malvaceae, Rosaceae, Saxifragaceae, Scrophulariaceae, Solanaceae, Tetragoniaceae.

Annual, perennial, monocotyledonous and dicotyledonous plants are preferred host organisms for the generation of transgenic plants. The use of the recombination system, or method according to the invention is furthermore advantageous in all ornamental plants, forestry, fruit, or ornamental trees, flowers, cut flowers, shrubs or turf. Said plant may include—but shall not be limited to—bryophytes such as, for example, Hepaticae (hepaticas) and Musci (mosses); pteridophytes such as ferns, horsetail and clubmosses; gymnosperms such as conifers, cycads, ginkgo and Gnetaeae; algae such as Chlorophyceae, Phaeophpyceae, Rhodophyceae, Myxophyceae, Xanthophyceae, Bacillariophyceae (diatoms) and Euglenophyceae.

Plants for the purposes of the invention may comprise the families of the Rosaceae such as rose, Ericaceae such as rhododendrons and azaleas, Euphorbiaceae such as poinsettias and croton, Caryophyllaceae such as pinks, Solanaceae such as petunias, Gesneriaceae such as African violet, Balsaminaceae such as touch-me-not, Orchidaceae such as orchids, lridaceae such as gladioli, iris, freesia and crocus, Compositae such as marigold, Geraniaceae such as geraniums, Liliaceae such as drachaena, Moraceae such as ficus, Araceae such as philodendron and many others.

The transgenic plants according to the invention are furthermore selected in particular from among dicotyledonous crop plants such as, for example, from the families of the Leguminosae such as pea, alfalfa and soybean; the family of the Umbelliferae, particularly the genus Daucus (very particularly the species carota (carrot)) and Apium (very particularly the species graveolens dulce (celery)) and many others; the family of the Solanaceae, particularly the genus Lycopersicon, very particularly the species esculenturn (tomato) and the genus Solanum, very particularly the species tuberosum (potato) and melongena (aubergine), tobacco and many others; and the genus Capsicum, very particularly the species annum (pepper) and many others; the family of the Leguminosae, particularly the genus Glycine, very particularly the species max (soybean) and many others; and the family of the Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli); and the genus Arabidopsis, very particularly the species thaliana and many others; the family of the Compositae, particularly the genus Lactuca, very particularly the species sativa (lettuce) and many others.

The transgenic plants according to the invention are selected in particular among monocotyledonous crop plants, such as, for example, cereals such as wheat, barley, sorghum and millet, rye, triticale, maize, rice or oats, and sugar cane.

Further preferred are trees such as apple, pear, quince, plum, cherry, peach, nectarine, apricot, papaya, mango, and other woody species including coniferous and deciduous trees such as poplar, pine, sequoia, cedar, oak, etc.

Especially preferred are Arabidopsis thaliana, Nicotiana tabacum, oilseed rape, soybean, corn (maize), wheat, linseed, potato and tagetes.

Plant varieties may be excluded, particularly registrable plant varieties according to Plant Breeders Rights. It is noted that a plant need not be considered a “plant variety” simply because it contains stably within its genome a transgene, introduced into a cell of the plant or an ancestor thereof.

In addition to a plant, the present invention provides any clone of such a plant, seed, selfed or hybrid progeny and descendants, and any part or propagule of any of these, such as cuttings and seed, which may be used in reproduction or propagation, sexual or asexual. Also encompassed by the invention is a plant which is a sexually or asexually propagated off-spring, clone or descendant of such a plant, or any part or propagule of said plant, off-spring, clone or descendant.

Plant organisms are furthermore, for the purposes of the invention, other organisms which are capable of photosynthetic activity, such as, for example, algae or cyanobacteria, and also mosses. Preferred algae are green algae, such as, for example, algae of the genus Haematococcus, Phaedactylum tricornatum, Volvox or Dunaliella.

Genetically modified plants according to the invention which can be consumed by humans or animals can also be used as food or feedstuffs, for example directly or following processing known in the art.

### VI. Methods for Introducing Constructs into Target Cells

A DNA construct according to the invention may advantageously be introduced into cells using vectors into which said DNA construct is inserted. Examples of vectors may be plasmids, cosmids, phages, viruses, retroviruses or agrobacteria. In an advantageous embodiment, the expression cassette is introduced by means of plasmid vectors. Preferred vectors are those which enable the stable integration of the expression cassette into the host genome.

The DNA construct can be introduced into the target plant cells and/or organisms by any of the several means known to those of skill in the art, a procedure which is termed transformation (see also Keown et al. (1990) Meth Enzymol 185:527-537). Production of stable, fertile transgenic plants in almost all economically relevant monocot plants is now routine:(Toriyama, et al. (1988) Bio/Technology 6:1072-1074; Zhang et al. (1988) Plant Cell Rep. 7:379-384; Zhang, et al. (1988) Theor Appl Genet 76:835-840; Shimamoto et al. (1989) Nature 5338:274-276; Datta et al. (1990) Bio/Technology 8:736-740; Christou et al. (1991) Bio/Technology 9:957-962; Peng, et al. (1991) International Rice Research Institute, Manila, Philippines 563-574; Cao et al. (1992) Plant Cell Rep 11:585-591; Li et al. (1993) Plant Cell Rep. 12:250-255; Rathore et al. (1993) Plant Mol Biol 21:871-884; Fromm et al. (1990) Bio/Technology 8:833-839; Gordon-Kamm et al. (1990) Plant Cell 2:603-618; D'Halluin et al. (1992) Plant Cell 4:1495-1505; Walters et al. (1992) Plant Mol Biol 18:189-200; Koziel et al. (1993) Biotechnology 11:194-200; Vasil I K (1994) Plant Mol Biol 25, 925-937; Weeks et al. 11993) Plant Physiology 102, 1077-1084; Somers et al. (1992) Bio/Technology 10, 1589-1594; WO 92/14828).

For instance, the DNA constructs can be introduced into cells, either in culture or in the organs of a plant by a variety of conventional techniques. For example, the DNA constructs can be introduced directly to plant cells using ballistic methods, such as DNA particle bombardment, or the DNA construct can be introduced using techniques such as electroporation and microinjection of a cell. Particle-mediated transformation techniques (also known as “biolistics”) are described in, e.g., Klein et al. (1987) Nature 327:70-73; Vasil V et al. (1993) Bio/Technol 11:1553-1558; and Becker D et al. (1994) Plant J 5:299-307. These methods involve penetration of cells by small particles with the nucleic acid either within the matrix of small beads or particles, or on the surface. The biolistic PDS-1000 Gene Gun (Biorad, Hercules, Calif.) uses helium pressure to accelerate DNA-coated gold or tungsten microcarriers toward target cells. The process is applicable to a wide range of tissues and cells from organisms, including plants. Other transformation methods are also known to those of skill in the art.

Microinjection techniques are known in the art and are well described in the scientific and patent literature. Also, the cell can be permeabilized chemically, for example using polyethylene glycol, so that the DNA can enter the cell by diffusion. The DNA can also be introduced by protoplast fusion with other DNA-containing units such as minicells, cells, lysosomes or liposomes. The introduction of DNA constructs using polyethylene glycol (PEG) precipitation is described in Paszkowski et al. (1984) EMBO J 3:2717. Liposome-based gene delivery is e.g., described in WO 93/24640; Mannino and Gould-Fogerite (1988) BioTechniques 6(7):682-691; U.S. Pat. No. 5,279,833; WO 91/06309; and Feigner et al. (1987) Proc Natl Acad Sci USA 84:7413-7414).

Another suitable method of introducing DNA is electroporation, where the cells are permeabilized reversibly by an electrical pulse. Electroporation techniques are described in Fromm et al. (1985) Proc Natl Acad Sci USA 82:5824. PEG-mediated transformation and electroporation of plant protoplasts are also discussed in Lazzeri P (1995) Methods Mol Biol 49:95-106. Preferred general methods which may be mentioned are the calcium-phosphate-mediated transfection, the DEAE-dextran-mediated transfection, the cationic lipid-mediated transfection, electroporation, transduction and infection. Such methods are known to the skilled worker and described, for example, in Davis et al., Basic Methods In Molecular Biology (1986). For a review of gene transfer methods for plant and cell cultures, see, Fisk et al. (1993) Scientia Horticulturae 55:5-36 and Potrykus (1990) CIBA Found Symp 154:198.

Methods are known for introduction and expression of heterologous genes in both monocot and dicot plants. See, e.g., U.S. Pat. No. 5,633,446, U.S. Pat. No. 5,317,096, U.S. Pat. No. 5,689,052, U.S. Pat. No. 5,159,135, and U.S. Pat. No. 5,679,558; Weising et al. (1988) Ann. Rev. Genet. 22: 421-477. Transformation of monocots in particular can use various techniques including electroporation (e.g., Shimamoto et al. (1992) Nature 338:274-276; biolistics (e.g., EP-A1 270,356); and Agrobacterium (e.g., Bytebier et al. (1987) Proc Natl Acad Sci USA 84:5345-5349). In particular, Agrobacterium mediated transformation is now a highly efficient transformation method in monocots (Hiei et al. (1994) Plant J 6:271-282). Aspects of the invention provide an expression vector for use in such transformation methods which is a disarmed Agrobacterium Ti plasmid, and an Agrobacterium tumefaciens bacteria comprising such an expression vector. The generation of fertile transgenic plants has been achieved using this approach in the cereals rice, maize, wheat, oat, and barley (reviewed in Shimamoto K (1994) Current Opinion in Biotechnology 5:158-162; Vasil et al. (1992) Bio/Technology 10:667-674; Vain et al. (1995) Biotechnology Advances 13(4):653-671; Vasil (1996) Nature Biotechnology 14:702; Wan & Lemaux (1994) Plant Physiol. 104:37-48)

Other methods, such as microprojectile or particle bombardment (U.S. Pat. No. 5,100,792, EP-A-444 882, EP-A-434 616), electroporation (EP-A 290 395, WO 87/06614), microinjection (WO 92/09696, WO 94/00583, EP-A 331 083, EP-A 175 966, Green et al. (1987) Plant Tissue and Cell Culture, Academic Press) direct DNA uptake (DE 4005152, WO 90/12096, U.S. Pat. No. 4,684,611), liposome mediated DNA uptake (e.g. Freeman et al. (1984) Plant Cell Physiol 2 9:1353), or the vortexing method (e.g., Kindle (1990) Proc Natl Acad Sci USA 87:1228) may be preferred where Agrobacterium transformation is inefficient or ineffective.

In particular, transformation of gymnosperms, such as conifers, may be performed using particle bombardment 20 techniques (Clapham D et al. (2000) Scan J For Res 15: 151-160). Physical methods for the transformation of plant cells are reviewed in Oard, (1991) Biotech. Adv. 9 :1-11. Alternatively, a combination of different techniques may be employed to enhance the efficiency of the transformation process, e.g. bombardment with Agrobacterium coated microparticles (EP-A-486234) or microprojectile bombardment to induce wounding followed by co-cultivation with Agrobacterium (EP-A-486233).

In plants, methods for transforming and regenerating plants from plant tissues or plant cells with which the skilled worker is familiar are exploited for transient or stable transformation. Suitable methods are especially protoplast transformation by means of polyethylene-glycol-induced DNA uptake, biolistic methods such as the gene gun (“particle bombardment” method), electroporation, the incubation of dry embryos in DNA-containing solution, sonication and microinjection, and the transformation of intact cells or tissues by micro- or macroinjection into tissues or embryos, tissue electroporation, or vacuum infiltration of seeds. In the case of injection or electroporation of DNA into plant cells, the plasmid used does not need to meet any particular requirement. Simple plasmids such as those of the pUC series may be used. If intact plants are to be regenerated from the transformed cells, the presence of an additional selectable marker gene on the plasmid is useful.

In addition to these “direct” transformation techniques, transformation can also be carried out by bacterial infection by means of Agrobacterium tumefaciens or Agrobacterium rhizogenes. These strains contain a plasmid (Ti or Ri plasmid). Part of this plasmid, termed T-DNA (transferred DNA), is transferred to the plant following agrobacterial infection and integrated into the genome of the plant cell.

For Agrobacterium-mediated transformation of plants, the DNA construct of the invention may be combined with suitable T-DNA flanking regions and introduced into a conventional Agrobacterium tumefaciens host vector. The virulence functions of the A. tumefaciens host will direct the insertion of a transgene and adjacent marker gene(s) (if present) into the plant cell DNA when the cell is infected by the bacteria. Agrobacterium tumefaciens-mediated transformation techniques are well described in the scientific literature. See, for example, Horsch et al. (1984) Science 233:496-498, Fraley et al. (1983) Proc Natl Acad Sci USA 80:4803-4807, Hooykaas (1989) Plant Mol Biol 13:327-336, Horsch R B (1986) Proc Natl Acad Sci USA 83(8):2571-2575), Bevans et al. (1983) Nature 304:184-187, Bechtold et al. (1993) Comptes Rendus De L'Academie: Des Sciences Serie III-Sciences De La Vie-Life Sciences 316:1194-1199, Valvekens et al. (1988) Proc Natl Acad Sci USA 85:5536-5540.

The DNA construct is preferably integrated into specific plasmids, either into a shuttle, or intermediate, vector or into a binary vector). If, for example, a Ti or Ri plasmid is to be used for the transformation, at least the right border, but in most cases the right and the left border, of the Ti or Ri plasmid T-DNA is linked with the expression cassette to be introduced as a flanking region. Binary vectors are preferably used. Binary vectors are capable of replication both in E. coli and in Agrobacterium. As a rule, they contain a selection marker gene and a linker or polylinker flanked by the right or left T-DNA flanking sequence. They can be transformed directly into Agrobacterium (Holsters et al. (1978) Mol Gen Genet 163:181-187). The selection marker gene permits the selection of transformed agrobacteria and is, for example, the DAAO gene of the invention, which imparts resistance to—for example—D-alanine or D-serine. The agrobacterium, which acts as host organism in this case, should already contain a plasmid with the vir region. The latter is required for transferring the T-DNA to the plant cell. An agrobacterium thus transformed can be used for transforming plant cells.

Many strains of Agrobacterium tumefaciens are capable of transferring genetic material —for example the DNA construct according to the invention—, such as, for example, the strains EHA101[pEHA101] (Hood E E et al. (1996) J Bacteriol 168(3):1291-1301), EHA105[pEHA105] (Hood et al. 1993, Transgenic Research 2, 208-218), LBA4404[pAL4404] (Hoekema et al. (1983) Nature 303:179-181), C58C1[pMP90] (Koncz and Schell (1986) Mol Gen Genet 204,383-396) and C58C1[pGV2260] (Deblaere et al. (1985) Nucl Acids Res. 13, 4777-4788).

The agrobacterial strain employed for the transformation comprises, in addition to its disarmed Ti plasmid, a binary plasmid with the T-DNA to be transferred, which, as a rule, comprises a gene for the selection of the transformed cells and the gene to be transferred. Both genes must be equipped with transcriptional and translational initiation and termination signals. The binary plasmid can be transferred into the agrobacterial strain for example by electroporation or other transformation methods (Mozo & Hooykaas (1991). Plant Mol Biol 16:917-918). Co-culture of the plant explants with the agrobacterial strain is usually performed for two to three days.

A variety of vectors could, or can, be used. In principle, one differentiates between those vectors which can be employed for the agrobacterium-mediated transformation or agroinfection, i.e. which comprise the DNA construct of the invention within a T-DNA, which indeed permits stable integration of the T-DNA into the plant genome. Moreover, border-sequence-free vectors may be employed, which can be transformed into the plant cells for example by particle bombardment, where they can lead both to transient and to stable expression.

The use of T-DNA for the transformation of plant cells has been studied and described intensively (EP-A1 120 516; Hoekema, In: The Binary Plant Vector System, Offsetdrukkerij Kanters B. V., Alblasserdam, Chapter V; Fraley et al. (1985) Crit Rev Plant Sci 4:1-45 and An et al. (1985) EMBO J 4:277-287). Various binary vectors are known, some of which are commercially available such as, for example, pBIN19 (Clontech Laboratories, Inc. USA).

To transfer the DNA to the plant cell, plant explants are cocultured with Agrobacterium tumefaciens or Agrobacterium rhizogenes. Starting from infected plant material (for example leaf, root or stalk sections, but also protoplasts or suspensions of plant cells), intact plants can be regenerated using a suitable medium which may contain, for example, antibiotics or biocides for selecting transformed cells. The plants obtained can then be screened in the presence of the DNA introduced, in this case the DNA construct according to the invention. As soon as the DNA has integrated into the host genome, the genotype in question is, as a rule, stable and the insertion in question is also found in the subsequent generations. Preferably the stably transformed plant is selected using the method of the invention (however other selection schemes employing other selection markers comprised in the DNA construct of the invention may be used). The plants obtained can be cultured and hybridized in the customary fashion. Two or more generations should be grown in order to ensure that the genomic integration is stable and hereditary.

The abovementioned methods are described, for example, in B. Jenes et al., Techniques for Gene Transfer, in: Transgenic Plants, Vol. 1, Engineering and Utilization, edited by S D Kung and R Wu, Academic Press (1993), 128-143 and in Potrykus (1991) Annu Rev Plant Physiol Plant Molec Biol 42:205-225). The construct to be expressed is preferably cloned into a vector which is suitable for the transformation of Agrobacterium tumefaciens, for example pBin19 (Bevan et al. (1984) Nucl Acids Res 12:8711).

The DNA construct of the invention can be used to confer desired traits on essentially any plant. One of skill will recognize that after DNA construct is stably incorporated in transgenic plants and confirmed to be operable, it can be introduced into other plants by sexual crossing. Any of a number of standard breeding techniques can be used, depending upon the species to be crossed.

### VII. Regeneration of Transgenic Plants

Transformed cells, i.e. those which comprise the DNA integrated into the DNA of the host cell, can be selected from untransformed cells preferably using the selection method of the invention. As soon as a transformed plant cell has been generated, an intact plant can be obtained using methods known to the skilled worker. For example, callus cultures are used as starting material. The formation of shoot and root can be induced in this as yet undifferentiated cell biomass in the known fashion. The shoots obtained can be planted and cultured.

Transformed plant cells, derived by any of the above transformation techniques, can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124176, Macmillian Publishing Company, N.Y. (1983); and in Binding, Regeneration of Plants, Plant Protoplasts, pp. 21-73, CRC Press, Boca Raton (1985). Regeneration can also be obtained from plant callus, explants, somatic embryos (Dandekar et al. (1989) J Tissue Cult Meth 12:145; McGranahan et al. (1990) Plant Cell Rep 8:512), organs, or parts thereof. Such regeneration techniques are described generally in Klee et al. (1987) Ann Rev Plant Physiol 38:467-486. Other available regeneration techniques are reviewed in Vasil et al., Cell Culture and Somatic Cell Genetics of Plants , Vol I, II, and III, Laboratory Procedures and Their Applications, Academic Press, 1984, and Weissbach and Weissbach, Methods for Plant Molecular Biology, Academic Press, 1989.

### VIII. Generation of Descendants

After transformation, selection and regeneration of a transgenic plant (comprising the DNA construct of the invention) descendants are generated, which—because of the activity of the excision promoter—underwent excision and do not comprise the marker sequence(s) and expression cassette for the endonuclease.

Descendants can be generated by sexual or non-sexual propagation. Non-sexual propagation can be realized by introduction of somatic embryogenesis by techniques well known in the art. Preferably, descendants are generated by sexual propagation/fertilization. Fertilization can be realized either by selfing (self-pollination) or crossing with other transgenic or non-transgenic plants. The transgenic plant of the invention can herein function either as maternal or paternal plant.

After the fertilization process, seeds are harvested, germinated and grown into mature plants. Isolation and identification of descendants which underwent the excision process can be done at any stage of plant development. Methods for said identification are well known in the art and may comprise—for example—PCR analysis, Northern blot, Southern blot, or phenotypic screening (e.g., for an negative selection marker).

Descendants may comprise one or more copies of the agronomically valuable trait gene. Preferably, descendants are isolated which only comprise one copy of said trait gene.

In a preferred embodiment the transgenic plant made by the process of the invention is marker-free. The terms “marker-free” or “selection marker free” as used herein with respect to a cell or an organisms are intended to mean a cell or an organism which is not able to express a functional selection marker protein (encoded by expression cassette b; as defined above) which was inserted into said cell or organism in combination with the gene encoding for the agronomically valuable trait. The sequence encoding said selection marker protein may be absent in part or—preferably—entirely. Furthermore the promoter operably linked thereto may be dysfunctional by being absent in part or entirely.

The resulting plant may however comprise other sequences which may function as a selection marker. For example the plant may comprise as a agronomically valuable trait a herbicide resistance conferring gene. However, it is most preferred that the resulting plant does not comprise any selection marker.

Also in accordance with the invention are cells, cell cultures, parts—such as, for example, in the case of transgenic plant organisms, roots, leaves and the like—derived from the above-described transgenic organisms, and transgenic propagation material (such as seeds or fruits).

Genetically modified plants according to the invention which can be consumed by humans or animals can also be used as food or feedstuffs, for example directly or following processing known per se. Here, the deletion of, for example, resistances to antibiotics and/or herbicides, as are frequently introduced when generating the transgenic plants, makes sense for reasons of customer acceptance, but also product safety.

A further subject matter of the invention relates to the use of the above-described transgenic organisms according to the invention and the cells, cell cultures, parts—such as, for example, in the case of transgenic plant organisms, roots, leaves and the like—derived from them, and transgenic propagation material such as seeds or fruits, for the production of food or feedstuffs, pharmaceuticals or fine chemicals. Here again, the deletion of, for example, resistances to antibiotics and/or herbicides is advantageous for reasons of customer acceptance, but also product safety.

Fine chemicals is understood as meaning enzymes, vitamins, amino acids, sugars, fatty acids, natural and synthetic flavors, aromas and colorants. Especially preferred is the production of tocopherols and tocotrienols, and of carotenoids. Culturing the transformed host organisms, and isolation from the host organisms or from the culture medium, is performed by methods known to the skilled worker. The production of pharmaceuticals such as, for example, antibodies or vaccines, is described by Hood E E, Jilka J M. (1999) Curr Opin Biotechnol. 10(4):382-386; Ma J K and Vine N D (1999) Curr Top Microbiol Immunol. 236:275-92).

Various further aspects and embodiments of the present invention will be apparent to those skilled in the art in view of the present disclosure. All documents mentioned in this specification are incorporated herein in their entirety by reference. Certain aspects and embodiments of the invention will now be illustrated by way of example and with reference to the figure described below.

### IX. Sequences

| 1. SEQ ID NO: 1: | Nucleic acid sequence encoding D-amino acid oxidase from | | |
|---|---|---|---|
| | Rhodosporidium toruloides (Yeast) | | |
| 2. SEQ ID NO: 2: | Amino acid sequence encoding D-amino acid oxidase from | | |
| | Rhodosporidium toruloides (Yeast) | | |
| 3. SEQ ID NO: 3: | Nucleic acid sequence encoding D-amino acid oxidase from | | |
| | Caenorhabditis elegans | | |
| 4. SEQ ID NO: 4: | Amino acid sequence encoding D-amino acid oxidase from | | |
| | Caenorhabditis elegans | | |
| 5. SEQ ID NO: 5: | Nucleic acid sequence encoding D-amino acid oxidase from | | |
| | Nectria haematococca | | |
| 6. SEQ ID NO: 6: | Amino acid sequence encoding D-amino acid oxidase from | | |
| | Nectria haematococca | | |
| 7. SEQ ID NO: 7: | Nucleic acid sequence encoding D-amino acid oxidase from | | |
| | Trigonopsis variabilis | | |
| 8. SEQ ID NO: 8: | Amino acid sequence encoding D-amino acid oxidase from | | |
| | Trigonopsis variabilis | | |
| 9. SEQ ID NO: 9: | Nucleic acid sequence encoding D-amino acid oxidase from | | |
| | Schizosaccharomyces pombe (fission yeast) | | |
| 10. SEQ ID NO: 10: | Amino acid sequence encoding D-amino acid oxidase from | | |
| | Schizosaccharomyces pombe (fission yeast) | | |
| 11. SEQ ID NO: 11: | Nucleic acid sequence encoding D-amino acid oxidase from | | |
| | Streptomyces coelicolor A3(2) | | |
| 12. SEQ ID NO: 12: | Amino acid sequence encoding D-amino acid oxidase from | | |
| | Streptomyces coelicolor A3(2) | | |
| 13. SEQ ID NO: 13: | Nucleic acid sequence encoding D-amino acid oxidase from | | |
| | Candida boidinii | | |
| 14. SEQ ID NO: 14: | Amino acid sequence encoding D-amino acid oxidase from | | |
| | Candida boidinii | | |
| 15. SEQ ID NO: 15: | Nucleic acid sequence coding for expression vector STPT GUS | | |
| | Nit-P daao (circular plasmid; total length 12334 bp) | | |
| | Feature | Position (bp) | Orientation |
| | RB (Agrobacterium right border) | 38-183 | direct |
| | nos-T (Nos terminator) | 384-639 | complementary |
| | daao (R. gracilis DAAO) | 716-1822 | complementary |
| | nit 1 - P (nitrilase I promoter) | 1866-3677 | complementary |
| | 35SpA (35S terminator) | 3767-3971 | complementary |
| | GUS (int) (β-glucuronidase) | 4046-6043 | complementary |
| | STPT (sTPT promoter) | 6097-7414 | complementary |
| | LB (Agrobacterium left border) | 7486-7702 | direct |
| 16. SEQ ID NO: 16: | Nucleic acid sequence coding for expression vector STPT GUS | | |
| | ptxA daao (circular plasmid; total length 11385 bp) | | |
| | Feature | Position (bp) | Orientation |
| | RB (Agrobacterium right border) | 38-183 | direct |
| | nos-T (Nos terminator) | 384-639 | complementary |
| | daao (R. gracilis DAAO) | 716-1822 | complementary |
| | ptxA (ptxA promoter) | 1866-2728 | complementary |
| | 35pA (35S terminator) | 2818-3022 | complementary |
| | GUS (int) (β-glucuronidase) | 3097-5094 | complementary |
| | STPT (sTPT promoter) | 5148-6465 | complementary |
| | LB (Agrobacterium left border) | 6537-6753 | direct |

### X. BRIEF DESCRIPTION OF THE FIGURES

FIG. 1: Basic Principle of the dual-function selection marker

A mixed population consisting of wild-type, non-transgenic plants (gray color) and transgenic plants comprising the DAAO marker (black color) is treated with either D-alanine or D-isoleucine. While the toxic effect of D-alanine on non-transgenic plants is detoxified by the transgene-mediated conversion (thereby selectively killing the wild-type plantlets), the non-toxic D-isoleucine is converted by the same enzymatic mechanism into a phytotoxic compound (thereby selectively killing the transgenic plantlets).

FIG. 2: Wild-type Arabidopsis thaliana plantlets (left side) and transgenic plantlets comprising the dual function marker (DAAO gene from Rhodotorula gracilis) are treated with either 30 mM D-isoleucine (upper side) or 30 mM D-alanine (bottom side). A toxic effect of D-isoleucine on the transgenic plants and D-alanine on the wild-type plants, respectively, can be observed, while no severe damage can be detected on the respective other group, thereby allowing for clear distinguishing and easy selection of either transgenic or wild-type plants.

FIG. 3 Effect of various D-amino acids on plant growth.

Wild type Arabidopsis thaliana plantlets were grown on half-concentrated Murashige-Skoog medium (0.5% (wt/vol) sucrose, 0.8% (wt/vol) agar) supplemented with the indicated D-amino acid at either 3 mM (Panel A) or 30 mM (Panel B). While D-alanine and D-serine are imposing severe phytotoxic effects even at 3 mM concentrations no significant effects can be observed for D-isoleucine.

FIG. 4 D-amino acid dose responses of dao1 transgenic and wild-type A. thaliana. (a-d) Growth of dao1 transgenic line 3:7 (white), 10:7 (light gray), 13:4 (gray) and wild-type (black) plants, in fresh weight per plant, on media containing various concentrations of D-serine, D-alanine, D-isoleucine and D-valine in half-strength MS with 0.5% (wt/vol) sucrose and 0.8% (wt/vol) agar. Different concentration ranges were used for different ID-amino acids. The plants were grown for 10 d after germination under 16 h photoperiods at 24° C.; n=10±s.e.m., except for plants grown on D-isoleucine, where smaller Petri dishes were used, (n=6±s.e.m.).

(e-l) Photographs of dao1 transgenic line 10:7 (e-h) and wild-type plants (i-l), grown for 10 d on the highest concentrations of the D-amino acid shown in the respective graphs above. All pictures have the same magnification. FW, fresh weight.

FIG. 5 Alignment of the catalytic site of various D-amino acid oxidases

Multiple alignment of the catalytic site of various D-amino acid oxidases allows for determination of a characteristic sequence motif [LIVM]-[LIVM]-H*-[NHA]-Y-G-x-[GSA]-[GSA]-x-G-x₅-G-x-A which allows for easy identification of additional D-amino acid oxidases suitable to be employed within the method and DNA-constructs of the invention.

FIG. 6 Vector map of construct expression vector STPT GUS Nit-P daao (Seq ID NO: 15; circular plasmid; total length 12334 bp)

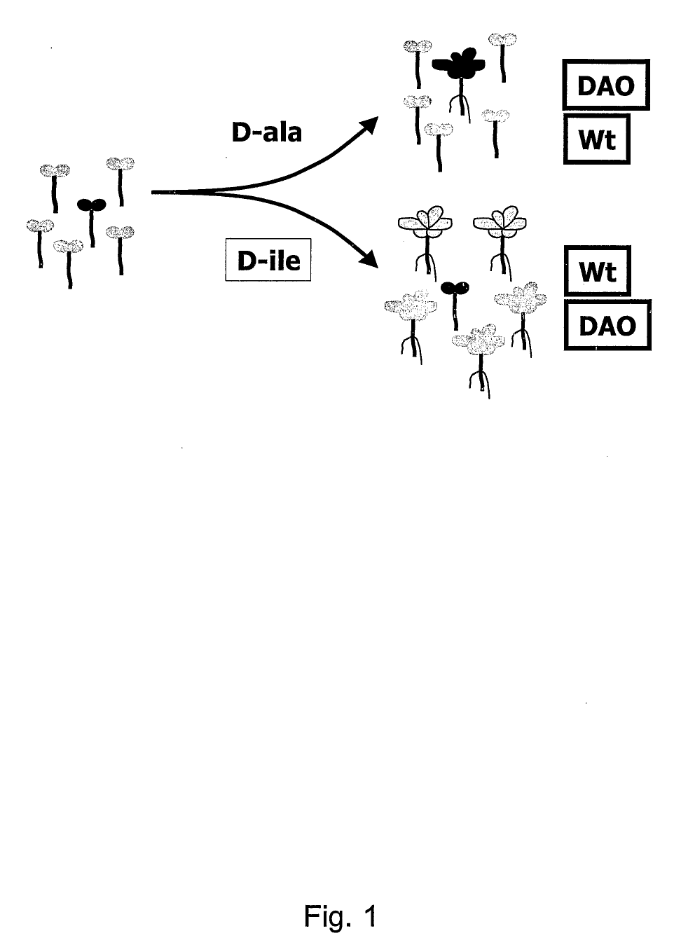

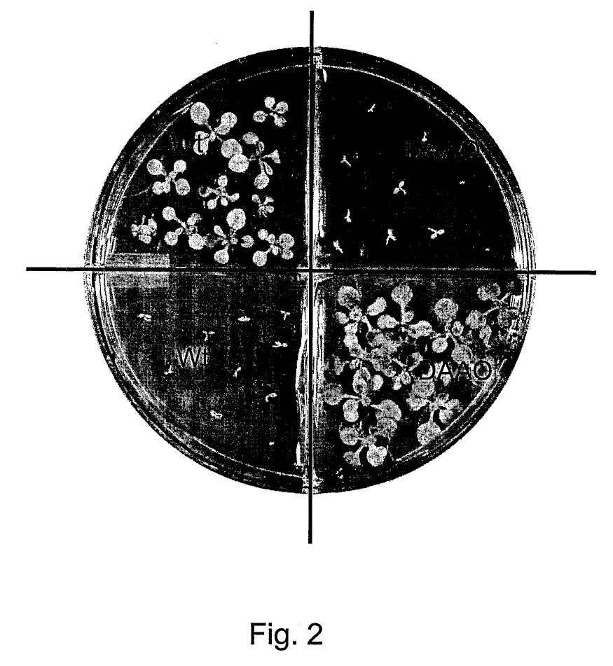

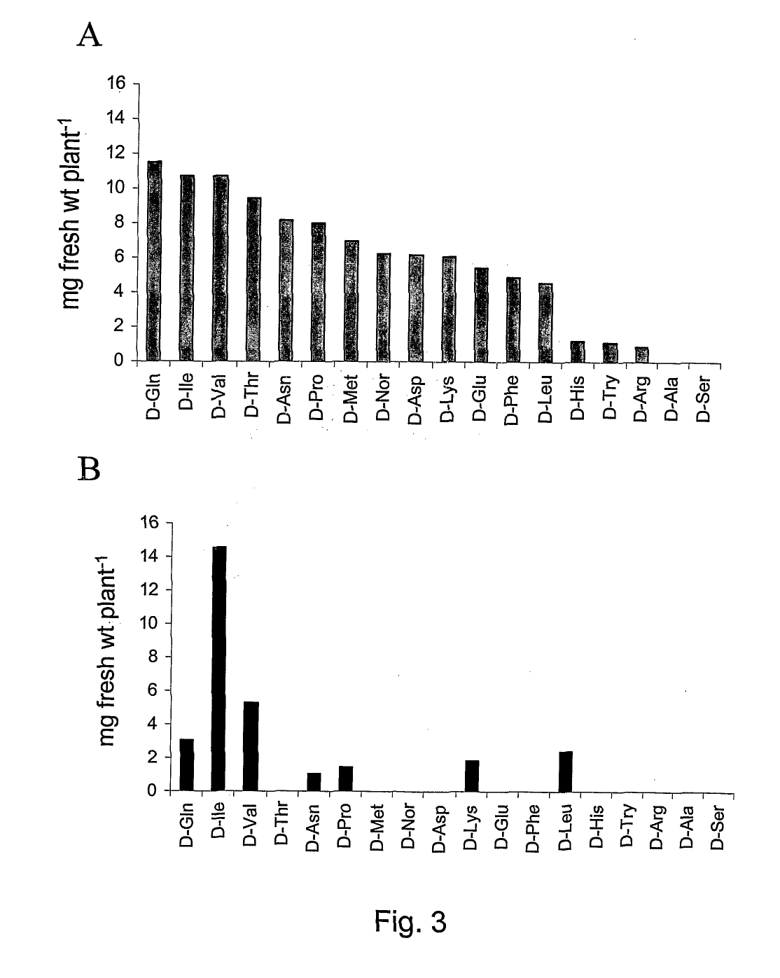

| Abbreviation | Feature | Position (bp) | Orientation |
|---|---|---|---|
| RB | Agrobacterium right border | 38-183 | direct |
| nos-T | Nos terminator | 384-639 | complementary |
| daao | R. gracilis DAAO | 716-1822 | complementary |
| nit 1 - P | nitrilase I promoter | 1866-3677 | complementary |
| 35SpA | 35S terminator | 3767-3971 | complementary |
| GUS (int) | β-glucuronidase | 4046-6043 | complementary |
| STPT | sTPT promoter | 6097-7414 | complementary |
| LB | Agrobacterium left border | 7486-7702 | direct |

| | |
|---|---|
| ColE1 | ColE1 origin of replication (E. coli) |
| aadA | Spectomycin/Strepotomycin resistance |
| repA/pVS1 | repA origin of replication (Agrobacterium) |

Furthermore, important restriction sites are indicated with their respective cutting position. The GUS gene is comrpising an intron (int).

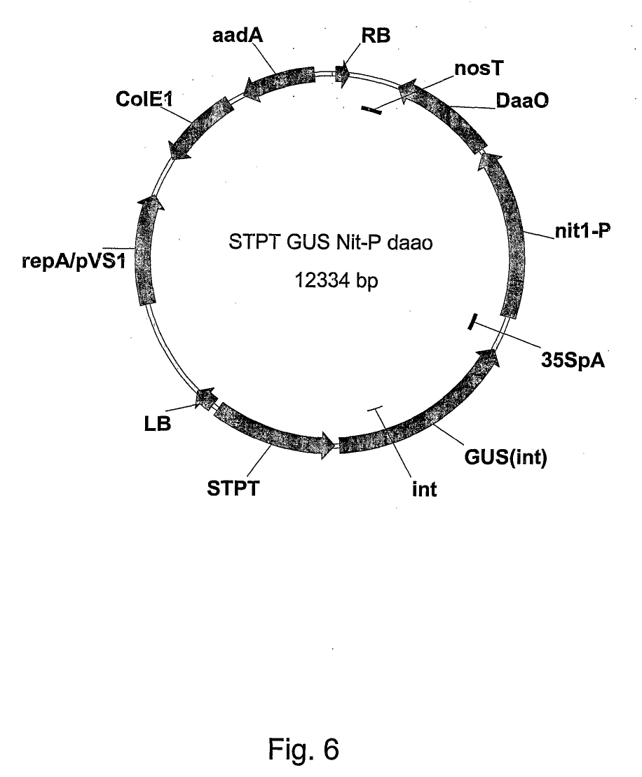

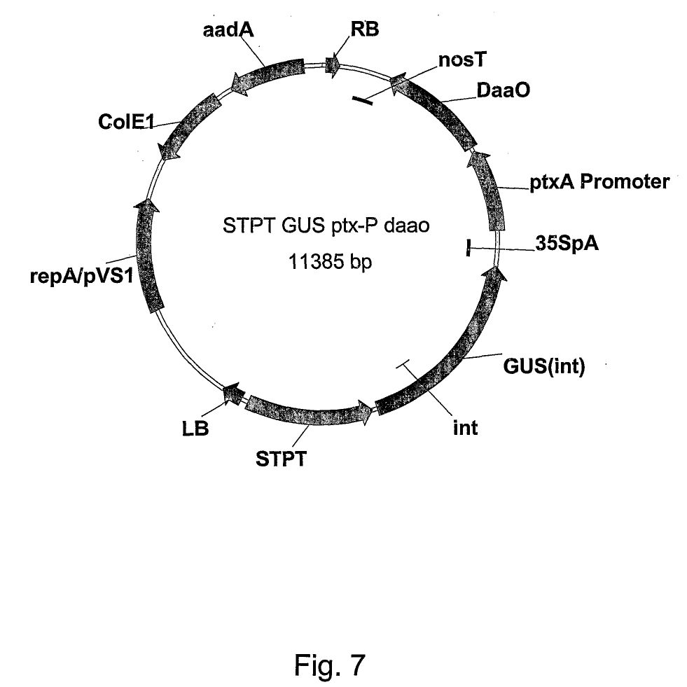

FIG. 6 Vector map of construct expression vector STPT GUS ptxA daao (SEQ ID NO: 16; circular plasmid; total length 11385 bp)

| Abbreviation | Feature | Position (bp) | Orientation |
|---|---|---|---|
| RB | Agrobacterium right border | 38-183 | direct |
| nos-T | Nos terminator | 384-639 | complementary |
| daao | R. gracilis DAAO | 716-1822 | complementary |
| ptxA | ptxA promoter | 1866-2728 | complementary |
| 35pA | 35S terminator | 2818-3022 | complementary |
| GUS (int) | β-glucuronidase | 3097-5094 | complementary |
| STPT | sTPT promoter | 5148-6465 | complementary |
| LB | Agrobacterium left border | 6537-6753 | direct |

| | |
|---|---|
| ColE1 | ColE1 origin of replication (E. coli) |
| aadA | Spectomycin/Strepotomycin resistance |
| repA/pVS1 | repA origin of replication (Agrobacterium) |

Furthermore, important restriction sites are indicated with their respective cutting position. The GUS gene is comrpising an intron (int).

### XI. EXAMPLES

General methods:

The chemical synthesis of oligonucleotides can be effected for example in the known manner using the phosphoamidite method (Voet, Voet, 2nd edition, Wiley Press N.Y., pages 896-897). The cloning steps carried out for the purposes of the present invention, such as, for example, restriction cleavages, agarose gel electrophoresis, purification of DNA fragments, the transfer of nucleic acids to nitrocellulose and nylon membranes, the linkage of DNA fragments, the transformation of E. coli cells, bacterial cultures, the propagation of phages and the sequence analysis of recombinant DNA are carried out as described by Sambrook et al. (1989) Cold Spring Harbor Laboratory Press; ISBN 0-87969-309-6. Recombinant DNA molecules were sequenced using an ALF Express laser fluorescence DNA sequencer (Pharmacia, Sweden) following the method of Sanger (Sanger et al., Proc. Natl. Acad. Sci. USA 74 (1977), 5463-5467).

### Example 1

### Vector Construction and Plant Transformation

DNA and RNA manipulation were done using standard techniques. The yeast R. gracillis was grown in liquid culture containing 30 mM D-alanine to induce dao1, the gene encoding DAAO. Total RNA was isolated from the yeast and used for cDNA synthesis. The PCR primers

5′-ATTAGATCTTACTACTCGAAGGACGCCATG-3′
and
5′-ATTAGATCTACAGCCACAATTCCCGCCCTA-3′

were used to amplify the dao1 gene from the cDNA template by PCR. The PCR fragments were sub-cloned into the pGEM-T Easy vector (Promega) and subsequently ligated into the BamHl site of the CaMV 35S expression cassette of the binary vector pPCV702kana17 giving pPCV702:dao1. The vectors were subjected to restriction analysis and sequencing to check that they contained the correct constructs.

### Example 1a

### Transformation of Arabidopsis Thaliana

A. thaliana plants (ecotype-Col-0) were grown in soil until they flowered. Agrobacterium tumefaciens (strain GV3101:pMP110 RK) transformed with the construct of interest was grown in 500 mL in liquid YEB medium (5 g/L Beef extract, 1 g/L Yeast Extract (Duchefa), 5 g/L Peptone (Duchefa), 5 g/L sucrose (Duchefa), 0,49 g/L MgSO₄ (Merck)) until the culture reached an OD₆₀₀ 0.8-1.0. The bacterial cells were harvested by centrifugation (15 minutes, 5,000 rpm) and resuspended in 500 mL infiltration solution (5% sucrose, 0.05% SILWET L-77 [distributed by Lehle seeds, Cat. No. VIS-02]).

Flowering A. thaliana plants were then transformed by the floral dip method (Clough S J & Bent A F (1998) Plant J. 16, 735-743 (1998) with the transgenic Agrobacterium tumefaciens strain carrying the vector described above by dipping for 10-20 seconds into the Agrobacterium solution. Afterwards the plants were kept in the greenhouse until seeds could be harvested. Transgenic seeds were selected by plating surface sterilized seeds on growth medium A (4.4 g/L MS salts [Sigma-Aldrich], 0.5 g/L MES [Duchefa]; 8 g/L Plant Agar [Duchefa]) supplemented with 50 mg/L kanamycin for plants carrying the nptII resistance marker, or 0.3 to 30 mM D-amino acids (as described below) for plants comprising the dual-function marker of the invention. Surviving plants were transferred to soil and grown in the greenhouse.

Lines containing a single T-DNA insertion locus were selected by statistical analysis of T-DNA segregation in the T2 population that germinated on kanamycin or D-amino acid -containing medium. Plants with a single locus of inserted T-DNA were grown and self-fertilized. Homozygous T3 seed stocks were then identified by analyzing T-DNA segregation in T3 progenies and confirmed to be expressing the introduced gene by northern blot analyses.

### Example 1b

### Agrobacterium-mediated Transformation of Brassica napus

Agrobacterium tumefaciens strain GV31 01 transformed with the plasmid of interest was grown in 50 mL YEB medium (see Example 4a) at 28° C. overnight. The Agrobacterium solution is mixed with liquid co-cultivation medium (double concentrated MSB5 salts (Duchefa), 30 g/L sucrose (Duchefa), 3.75 mg/l BAP (6-benzylamino purine, Duchefa), 0.5 g/l MES (Duchefa), 0.5 mg/l GA3 (Gibberellic Acid, Duchefa); pH 5.2) until OD₆₀₀ of 0.5 is reached. Petiols of 4 days old seedlings of Brassica napus cv. Westar grown on growth medium B (MSB5 salts (Duchefa), 3% sucrose (Duchefa), 0.8% oxoidagar (Oxoid GmbH); pH 5,8) are cut. Petiols are dipped for 2-3 seconds in the Agrobacterium solution and afterwards put into solid medium for co-cultivation (co-cultivation medium supplemented with 1.6% Oxoidagar). The co-cultivation lasts 3 days (at 24° C. and ˜50 μMol/m²s light intensity). Afterwards petiols are transferred to co-cultivation medium supplemented with the appropriate selection agent (18 mg/L kanamycin (Duchefa) for plants comprising the nptII marker kanamycin for plants carrying the nptII resistance marker; or 0.3 to 30 mM D-amino acids, as described below) for plants comprising the dual-function marker of the invention) and 300 mg/L Timetin (Duchefa)

Transformed petioles are incubated on the selection medium for four weeks at 24° C. This step is repeated until shoots appear. Shoots are transferred to A6 medium (MS salts (Sigma Aldrich), 20 g/L sucrose, 100 mg/L myo-inositol (Duchefa), 40 mg/L adeninesulfate (Sigma Aldrich), 500 mg/L MES, 0.0025 mg/L BAP (Sigma), 5 g/L oxoidagar (Oxoid GmbH), 150 mg/l timetin (Duchefa), 0.1 mg/L IBA (indol butyric acid, Duchefa); pH 5,8) supplemented with the appropriate selection agent (18 mg/L kanamycin (Duchefa) for plants comprising the nptII marker kanamycin for plants carrying the nptII resistance marker, or 0.3 to 30 mM D-amino acids; as described below) until they elongated. Elongated shoots are cultivated in A7 medium (A6 medium without BAP) for rooting. Rooted plants are transferred to soil and grown in the greenhouse.

### Example 1c

### Agrobacterium-mediated transformation of Zea Mays

Seeds of certain corn inbred lines or corn hybrid lines are germinated, rooted, and further grown in greenhouses. Ears from corn plants are harvested 8 to 14 (average 10) days after pollination (DAP) and immature embryos are isolated therefrom. Timing of harvest varies depending on growth conditions and maize variety. The optimal length of immature embryos for transformation is about 1 to 1.5 mm, including the length of the scutellum. The embryo should be translucent, not opaque. The excised embryos are collected in MS based liquid medium (comprising 1.5 mg/L 2,4-D). Acetosyringone (50 to 100 μM) is added to the medium at either the same time as inoculation with Agrobacterium or right before use for Agroinfection.

Preparation of Agrobacteria: Agrobacteria are grown on YEP medium. The Agrobacterium suspension is vortexed in the above indicated medium (comprising 100 μM acetosyringone media for preferably 1-2 hours prior to infection).

Inoculation/Co-cultivation: The bacterial suspension is added to the microtube (plate) containing pre-soaked immature embryos and left at room temperature (20-25° C.) for 5 to 30 min. Excess bacterial suspension is removed and the immature embryos and bacteria in the residue medium are transferred to a Petri plate. The immature embryos are placed on the co-cultivation medium with the flat side down (scutellum upward). The plate is sealed, and incubated in the dark at 22° C. for 2-3 days. (Co-cultivation medium: MS-base, 1.5 mg/l 2,4-D, 15 μM AgNO₃, 100 μM acetosyringone). Alternatively, excised immature embryos are directly put on the co-cultivation medium with the flat side down (scutellum upward). Diluted Agrobacterium cell suspension is added to each immature embryo. The plate is sealed, and incubated in the dark at 22° C. for 2-3 days.

Recovery: After co-cultivation the embryos are transferred to recovery media (MS-base comprising 1,5 mg/l 2,4-D, 150 mg/l Timentin), and incubate the plates in dark at 27° C. for about 5 to 7 days the scutellum side up.

Selection of transformed calli: The immature embryos are transferred to selection media (recovery medium further comprising the selective agent e.g., D-alanine in concentration of 0.3 to 30 mM) (scutellum up) and incubated in the dark at 27° C. for 10-14 days (First selection). All immature embryos that produce variable calli are subcultured to 2-3^{rd} selection media. At this stage, any roots that have formed are removed. Incubation occurs for 2 weeks under the same conditions for the first selection (Second selection). The regenerable calli is excised from the scutellum (the regenerable calli is whitish in color, compact, not slimy and may have some embryo-like structures) and transferred to fresh 2-3^{rd} selection media. Plates are wrapped and incubate in the dark at 27° C. for 2 weeks (3^{rd} selection may not be necessary for most of the genotypes, regenerable calli can be transferred to Regeneration medium).

Regeneration of transformed plants: Proliferated calli (whitish with embryonic structures forming) are excised in the same manner as for 2^{nd}/3^{rd} selection and transferred to regeneration media (like selection medium but without 2,4-D). Plates are wrapped and put in the light (ca. 2,000 lux) at 25 or 27° C. for 2 weeks, or until shoot-like structures are visible. Transfer to fresh regeneration media if necessary. Calli sections with regenerated shoots or shoot-like structures are transferred to a Phytatray containing rooting medium and incubate for 2 weeks under the same condition as above step, or until rooted plantlets have developed. After 2 to 4 weeks on rooting media (half-concentrated MS medium, no 2,4-D, no selective agent), calli that still have green regions (but which have not regenerated seedlings) are transferred to fresh rooting Phytatrays. Rooted seedlings are transferred to Metromix soil in greenhouse and covered each with plastic dome for at least 1 week, until seedlings have established. When plants reach the 3-4 leaf-stages, they are fertilized with Osmocote and then sprayed with selective agent (e.g., D-alanine or D-serine), and grown in the greenhouse for another two weeks. Non-transgenic plants should develop herbicidal symptoms or die in this time. Survived plants are transplanted into 10″ pots with MetroMix and 1 teaspoon Osmocote.

### Example 2

### Selection Analysis

T1 seeds of transgenic Arabidopsis plants were surface-sterilized and sown in Petri plates that were sealed with gas-permeable tape. The growth medium was half strength MS19 with 0.5% (wt/vol) sucrose and 0.8% (wt/vol) agar, plus 3 mM D-alanine, 3 mM D-serine or 50 μg/ml kanamycin as the selective agent. Plants were grown for 5 d after germination with a 16 h photoperiod at 24° C. To evaluate the selection efficiency on different substrates, 2,074, 1,914 and 1,810 T1 seeds were sown on D-alanine-, D-serine- and kanamycin-selective plates, respectively, and the number of surviving seedlings was counted (44, 32 and 43, respectively).

### Example 3

### Enzyme Assays

Soluble proteins were extracted by shaking 0.1 g samples of plant material that had been finely pulverized in a 1.5 ml Eppendorf tube in 1 ml of 0.1 M potassium phosphate buffer, pH 8. DAAO activity was then assayed as follows. Reaction mixtures were prepared containing 2,120 μl of 0.1 M potassium phosphate buffer, pH 8, 80 μl of crude protein extract and 100 μl of 0.3 M D-alanine. The samples were incubated for 2 h at 30° C. The enzyme activity was then assessed, by measuring the increase in absorbance at 220 nm (E=1.090 M⁻¹ cm⁻¹) associated with the conversion of D-alanine to pyruvate, after transferring the test tubes to boiling water for 10 to stop the reaction. In control reactions, D-alanine was added immediately before boiling. One unit of DAAO activity is defined as the turnover of one micromole of substrate per minute, and activity was expressed per gram plant biomass (fresh weight). The breakdown of D-isoleucine and D-valine in DAAO incubations, and the associated production of 3-methyl-2-oxopentanoate and 3-methyl-2-oxobutanoate, were analyzed by high-performance liquid chromatography. In other respects the reactions were carried out as described above.

### Example 4

### Dual-Function Selection Marker

The qualification of the DAAO enzyme as a dual-function selection marker was demonstrated by testing germinated T1 seeds on different selective media. The T-DNA contained both 35S:dao1 and pNos:nptII, allowing D-amino acid and kanamycin selection to be compared in the same lot of seeds.

T1 seeds were sown on medium containing kanamycin (50 μg/ml), D-alanine (3 mM) or D-serine (3 mM), and the transformation frequencies found on the different selective media were 2.37%, 2.12% and 1.67%, respectively. D-alanine had no negative effect on the transgenic plants, even at a concentration of 30 mM, but at this concentration, D-serine induced significant growth inhibition. Fewer transgenic plants were found after selection on 3 mM D-serine because the compound slightly inhibited the growth of the transgenic plants at this concentration.

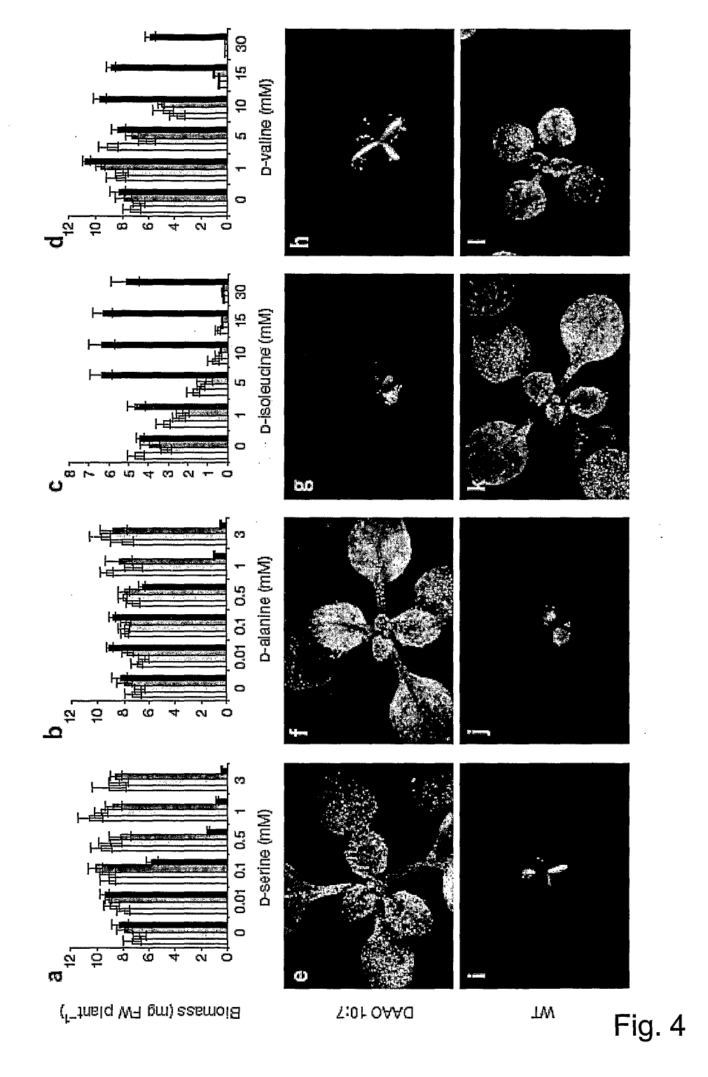

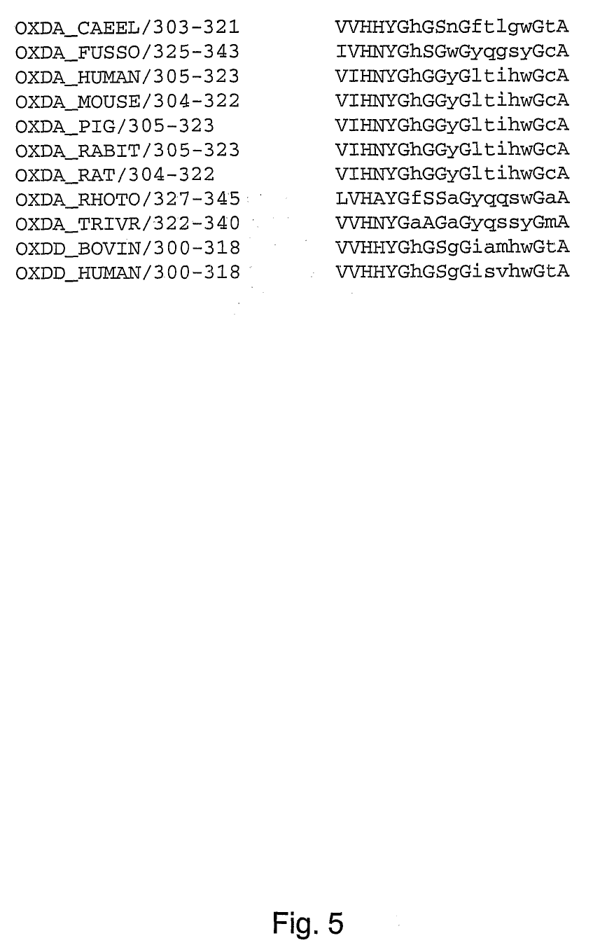

Further studies using lower concentrations corroborated this conclusion, and efficient selection using D-serine was achieved on concentrations lower than 1 mM (FIG. 4a). Progeny from the transgenic lines selected on D-serine and D-alanine were later confirmed to be kanamycin resistant, hence ensuring there would be no wild-type escapes from these lines.

Selection of seedlings on media containing D-alanine or D-serine was very rapid compared to selection on kanamycin. These D-amino acids inhibited growth of wild-type plants immediately after the cotyledons of wild-type plants had emerged. Therefore, transformants could be distinguished from non-transformed plants directly after germination. The difference between wild-type and transgenic plants after D-amino acid selection was unambiguous, with no intermediate phenotypes. In contrast, intermediate phenotypes are common when kanamycin resistance is used as a selection marker. Furthermore, wild-type seedlings were found to be sensitive to sprayed applications of D-serine and D-alanine. One-week-old seedlings were effectively killed when sprayed on three consecutive days with either 50 mM. D-serine or D-alanine, although the sensitivity of wild-type plants rapidly decreased with age, presumably because as the cuticle and leaves became thicker, uptake by the leaves was reduced. Transgenic seedlings were resistant to foliar application of D-alanine or D-serine, so selection on soil was possible.

Transgenic plants grown under D-alanine and D-serine selection conditions developed normally. Early development of transgenic plants from line 3:7, 10:7 and 13:4 was compared with that of wild-type plants by cultivation on vertical agar plates. No differences in biomass, number of leaves, root length or root architecture were detected for the different sets of plants. Furthermore, soil-cultivated wild-type and transgenic plants (line 10:7) showed no differences in the total number of rosette leaves, number of inflorescences and number of siliqua after 4 weeks of growth.

Also, the phenotypes of 17 individual T1 lines, which were picked for T-DNA segregation, were studied and found indistinguishable from that of wild type when grown on soil. A problem sometimes encountered after selection on antibiotics is the growth lag displayed by transformants. This phenomenon is explained as an inhibitory effect of the antibiotic on the transgenic plants (Lindsey K & Gallois P (1990) J. Exp. Bot 41, 529-536). However, unlike seedlings picked from antibiotic selection plates, transgenic seedlings picked from D-amino acid selection plates and transferred to soil were not hampered in their growth and development, even temporarily. A possible reason for this difference is that the DAAO scavenging of D-amino acids may effectively remove the D-amino acid in the plants. Furthermore, D-alanine and D-serine may merely provide additional growth substrates, because their catabolic products are carbon and nitrogen compounds that are central compounds in plant metabolism. Quantification of dao1 mRNA from six independent D-alanine- and D-serine-resistant lines showed a range of different expression levels mirrored in a range of different DAAO activities. In spite of these differences in mRNA levels and enzyme activities, no phenotypic variation associated with the D-serine and D-alanine treatment was found, suggesting that the DAAO marker is effective over a range of expression levels. As described above, D-isoleucine and D-valine were found to inhibit growth of the transgenic plants, but not the wild-type plants.

Therefore, plants containing the construct were tested as described above on two sets of media, one containing D-isoleucine and the other containing D-valine at various concentrations, to assess whether DAAO could also be used as a counter-selection marker. Unambiguous counter-selection selection was achieved when seeds were sown on either D-isoleucine or D-valine at concentrations greater than 10 mM (FIG. 4 c,d).

Thirteen individual lines expressing DAAO were tested for their response to D-isoleucine and all of them were effectively killed, whereas wild-type plants grew well, with no sign of toxicity. Similar results were obtained for D-valine, although this compound was found to have a moderately negative effect on wild-type plants at higher concentrations (FIG. 4d). The keto acid produced in DAAO catabolism of D-isoleucine is the same as that formed when L-isoleucine is metabolized by the endogenous branched-chain amino acid transaminase [EC: 2.6.1.42], namely 3-methyl-2-oxopentanoate (Kyoto Encyclopedia of Genes and Genomes, metabolic pathway web-site, http://www.genome.ad.jp/kegg/metabolism.html).

Presumably endogenous transaminase may be specific for the L-enantiomer, so the corresponding D-enantiomer is not metabolized in wildtype plants, but only in plants expressing DAAO. The negative effects of L-isoleucine (but not of the D-form) observed on wildtype plants, supports this speculation. Incubation of cell-free extracts from dao1 transgenic line 10:7 with D-isoleucine and D-valine resulted in 15-fold and 7-fold increases in production of 3-methyl-2-oxopentanoate and 3-methyl-2-oxobutanoate, respectively, compared to extracts of wild-type plants. Further, 3-methyl-2-oxopentanoate and 3- methyl-2-oxobutanoate impaired growth of A. thaliana, corroborating the suggestion that these compounds, or products of their metabolism, are responsible for the negative effects of D-isoleucine and D-valine on the transgenic plants. The toxicity of some D-amino acids on organisms is not well understood, and has only occasionally been studied in plants (Gamburg K Z & Rekoslavskaya N I (1991) Fiziologiya Rastenii 38, 1236-1246). Apart from A. thaliana, we have also tested the susceptibility of other plant species to D-serine, including poplar, tobacco, barley, maize, tomato and spruce. We found all tested species susceptible to D-serine at concentrations similar to those shown to be toxic for A. thaliana. A proposed mechanism for D-serine toxicity in bacteria is competitive inhibition of a-alanine coupling to pantoic acid, thus inhibiting formation of pantothenic acid (Cosloy S D & McFall E (1973) J. Bacteriol. 114, 685-694). It is possible to alleviate D-serine toxicity in D-serine-sensitive strains of Escherichia coli by providing pantothenic acid or a-alanine in the medium, but D-serine toxicity in A. thaliana could not be mitigated using these compounds. A second putative cause of D-amino acid toxicity is through competitive binding to tRNA. Knockout studies of the gene encoding D-Tyr-tRNATyr deacylase in E. coli have shown that the toxicity of D-tyrosine increases in the absence of deacylase activity (Soutourina J et al. (1996) J. Biol. Chem. 274, 19109-19114), indicating that D-amino acids interfere at the tRNA level. Genes similar to that encoding bacterial deacylase have also been identified. in A. thaliana (Soutourina J et al. (1996) J. Biol. Chem. 274, 19109-19114), corroborating the possibility that the mode of toxic action of D-amino acids might be through competitive binding to tRNA.

### Example 5

### Constructs Useful for Carrying Out the Invention

Two expression constructs are constructed for carrying out the present invention (SEQ ID NO: 15, 16). The backbone of both plasmid constructs (pSUN derivative) contains origins for the propagation in E. coli as well as in Agrobacterium and an aadA expression cassette (conferring spectinomycin and streptomycin resistance) to select for transgenic bacteria cells. The sequences for constructing the DNA constructs are amplified incorporating the appropriate restriction sites for subsequent cloning by PCR. Cloning was done by standard methods as described above. The sequence of the constructs is verified by DNA sequence analysis.

The first DNA construct (SEQ ID NO: 15) comprises an expression cassette for the D-amino acid oxidase (DAAO) from Rhodotorula gracilis under control of the Arabidopsis thaliana Nitrilase promoter (SEQ ID NO: 15; base pair 1866-3677, complementary orientation). Further comprised is an expression cassette for the β-glucuronidase which may function as a substitute for an agronomically valuable trait under control of the Arabidopsis sTPT promoter (i.e. TPT promoter truncated version, WO 03/006660; SEQ ID NO: 27 cited therein), and the CaMV 35S terminator.

The second DNA construct (SEQ ID NO: 16) comprises an expression cassette for the D-amino acid oxidase (DAAO) from Rhodotorula gracilis under control of the Pisum sativum ptxA promoter (SEQ ID NO: 16; base pair 1866-2728, complementary orientation). Further comprised is an expression cassette for the β-glucuronidase which may function as a substitute for an agronomically valuable trait under control of the Arabidopsis sTPT promoter (i.e. TPT promoter truncated version, WO 03/006660; SEQ ID NO: 27 cited therein), and the CaMV 35S terminator.

Transgenic Arabidopsis, Brassica napus, and Zea mays plants are generated as described above using either construct I (SEQ ID NO: 15) or construct II (SEQ ID NO: 16) for Agrobacterium mediated transformation. Transgenic plants are selected using the negative selection marker property of the D-amino acid oxidase on medium comprising 0.3, 3 or 30 mM D-alanine (or D-serine). Resulting transgenic plants are selfed to obtain homozygous plants. Homozygous plants are propagated over 2 to 3 generations to ensure stability of the transgenic insertion.

Seeds of transgenic plants are mixed with seeds of the corresponding non-transgenic line (used for transformation). Various proportions of transgenic versus non-transgenic seeds are used (1:1, 1:10, 1:100).

Seeds are sown on standard soil under green-house conditions. After germination, developing plantlets were sprayed at various developmental steps with preparations of D-isoleucine (final concentration of 10 mM, 20 mM, 30 mM, respectively in isotonic salt solution, pH 7.0).

None of the transgenic plants (detectable by GUS staining) is able to reach maturity under the above described conditions, while non-transgenic plants are unaffected by the treatment. Alternatively solutions of racemic D/L-isoleucine can be employed.

1. A method for preventing and/or suppressing growth of transgenic plants, which were grown on a field, in subsequent seasons among a population of other plants on said field or neighboring fields comprising the steps of:
i) providing seeds of a transgenic plant comprising at least one first expression cassette comprising a nucleic acid sequence encoding a D-amino acid oxidase operably linked with a promoter allowing expression in plants, in combination with at least one second expression cassette suitable for conferring to said plant an agronomically valuable trait, and
ii) in a first season sowing said seeds on a field, growing said transgenic plants, and harvesting the resulting plant products,
iii) providing at least one compound M, which is non-phytotoxic or moderately phytotoxic against plants not comprising a transgenic expression cassette for a D-amino acid oxidase, wherein said compound M can be metabolized by said D-amino acid oxidase into one or more compound(s) N which are phytotoxic or more phytotoxic than compound M, and
iii) in a subsequent season preventing and/or suppressing growth of said transgenic plants on said field or neighboring fields or areas, where other plants are grown or growing not comprising a transgenic expression cassette for a D-amino acid oxidase, by treating said fields or areas with said compound M in a concentration, which is non-phytotoxic against said other plants, but which is—in consequence of the metabolization into compound(s) N —phytotoxic against said transgenic plants thereby selectively preventing or suppressing growth of said transgenic plants.

2. The method of claim 1 wherein said compound M is comprises a D-amino acid structure selected from the group consisting of D-isoleucine, D-valine, D-asparagine, D-leucine, D-lysine, D-proline, and D-glutamine, and derivatives thereof.

3. The method of claim 1, wherein said compound M is selected from the group consisting of D-isoleucine and D-valine.

4. The method of any of claim 1, wherein said D-amino acid oxidase expressed from said first expression cassette has preferably metabolizing activity against at least one D-amino acid and comprises the following consensus sequence:
(SEQ ID NO: 17)
[LIVM]-[LIVM]-H*-[NHA]-Y-G-x-
[GSA]-[GSA]-x-G-x₅-G-x-A
wherein the amino acid residues given in brackets represent alternative residues for the respective position, x represents any amino acid residue, and indices numbers indicate the respective number of consecutive amino acid residues.

5. The method of claim 1, wherein said D-amino acid oxidase is described by a sequence of the group consisting of sequences described by GenBank or SwisProt Acc. No. JX01739, O33145, O35078, O45307, P00371, P14920, P18894, P22942, P24552, P31228, P80324, Q19564, Q28382, Q7PWX4, Q7PWY8, Q7Q7G4, Q7SFW4, Q7Z312, Q82MI8, Q86JV2, Q8N552, Q8P4M9, Q8PG95, Q8R2R2, Q8SZN5, Q8VCW7, Q921M5, Q922Z0, Q95XG9, Q99042, Q99489, Q9C1L2, Q9JXF8, Q9V5P1, Q9VM80, Q9X7P6, Q9Y7N4, Q9Z1M5, Q9Z302, and U60066.

6. The method of claim 1, wherein said D-amino acid oxidase is selected from the group of amino acid sequences consisting of
a) the sequences described by SEQ ID NO: 2, 4, 6, 8, 10, 12, and 14,
b) the sequences having a sequence homology of at least 40% with a sequence as described by SEQ ID NO: 2, 4, 6, 8, 10, 12, and 14, and
c) the sequences hybridizing under low or high stringency conditions with a sequence as described by SEQ ID NO: 2, 4, 6, 8, 10, 12, and 14.

7. A selective herbicidal composition comprising at least one compound M, wherein the compound M comprises a D-amino acid structure selected from the group consisting of D-isoleucine, D-valine, D-asparagine, D-leucine, D-lysine, D-proline, D-glutamine, and derivatives thereof.

8. The selective herbicidal composition of claim 7, comprising at least one compound selected from the group consisting of D-isoleucine, D-valine, and derivatives thereof.

9. A method of preventing or suppressing unwanted growth of transgenic plants comprising applying the compound M as defined in claim 7.

10. A method of preventing or suppressing unwanted growth of transgenic plants comprising applying the selective herbicide composition of claim 7.

11. A method of preventing or suppressing unwanted growth of transgenic plants comprising applying the selective herbicide composition of claim 8.
